(19) United States
(12) Patent Application Publication
(10) Pub. No.: US 2021/0106319 A1
(43) Pub. Date: Apr. 15, 2021

(54) CARDIAC CLOSURE DEVICE AND DELIVERY TOOL

(71) Applicant: NOVOGATE LTD., Givat Shmuel (IL)

(72) Inventors: Gil BOLOTIN, Zichron Yaakov (IL); Jaffar HLEIHIL, Jish (IL)

(21) Appl. No.: 16/464,515

(22) PCT Filed: Nov. 21, 2017
(86) PCT No.: PCT/IB2017/057307

§ 371 Date: May 28, 2019

Related U.S. Application Data
(60) Provisional application No. 62/426,339, filed on Nov. 25, 2016.

(51) Int. Cl. A61B 17/00

(52) U.S. Cl. CPC A61B 17/0057; A61B 2017/00579; A61B 2017/00867; A61B 2017/00592; A61B 2017/00623

(57) ABSTRACT

A cardiac closure device, apparatus and delivery tool are herein provided, wherein the apparatus includes an implantable cardiac closure device having a stretched state and a resting state, the closure device being configured for facilitating closure of a hole made through tissue of a myocardial wall of a patient; and a delivery tool comprising: a closure-device holder, at a distal portion of the delivery tool, being configured to hold the closure device, the closure-device holder being moveable proximally and distally along a longitudinal axis of the delivery tool; and one or more closure-device-shape-adjusting elements coupled to the closure-device holder, the one or more closure-device-shape-adjusting elements each being shaped so as to define a coupling for removably coupling the closure device to the one or more closure-device-shape-adjusting elements, the one or more closure-device-shape-adjusting elements being configured to adjust a shape of the closure device by being moveable radially inwardly toward the longitudinal axis of the delivery tool to transition the closure device from the stretched state toward the resting state.

### CROSS REFERENCE TO RELATED APPLICATIONS

This application claims the benefit of PCT Patent Application PCT/IB2017/057307, filed 21 Nov. 2017, entitled “CARDIAC CLOSURE DEVICE AND DELIVERY TOOL”, which is incorporated in its entirety herein by reference.

This application further claims priority from U.S. Provisional Patent Application No. 62/426,339, filed 25 Nov. 2016, entitled “CARDIAC CLOSURE DEVICE AND DELIVERY TOOL”, which is incorporated in its entirety herein by reference.

### FIELD OF THE APPLICATION

The present invention relates generally to cardiac surgical methods and devices, and specifically to minimally-invasive surgical tools and methods for performing transapical surgical procedures.

### BACKGROUND OF THE APPLICATION

Various cardiac medical procedures are performed using transapical delivery of medical devices to the left or right ventricle. The ventricle is accessed directly through a passage formed through the myocardium near the apex of ventricle. Such medical procedures include valve replacement, such as aortic or mitral valve replacement, and valve repair, such as mitral valve repair. Conventional transapical delivery procedures typically are performed under general anesthesia, and include performing a small thoracotomy, spreading the ribs using a mechanical retractor, opening of the pericardial sac, suturing the hole made through the ventricle, and closing the thoracotomy.

### SUMMARY OF APPLICATIONS

In some applications of the present invention, a closure device comprises a base and a plurality of anchors coupled to the base. The closure device is configured to assume at least a stretched state and a resting state. During a cardiac medical procedure, a surgeon couples the closure device to an external surface of the myocardium, by inserting anchoring portions of the anchors into tissue of the myocardium while the closure device is in the stretched state. The surgeon punctures the myocardium through the closure device to form a passage through the myocardium, and inserts a catheter into the heart via the closure device and the passage. After performing a medical procedure on the heart via the catheter, the surgeon withdraws the catheter from the heart. The surgeon causes the closure device to assume the resting state. Assumption of the resting state draws the anchors toward a central region of the base of the closure device, thereby squeezing together the cardiac tissue of the myocardium surrounding the passage made through the myocardium, and closing the passage.

For some applications, when the closure device assumes the resting state, the base of the closure device is shaped so as to define: (a) two or more inwardly-extending portions, which extend toward a central region of the base of the closure device, and (b) two or more outwardly-extending portions, which extend away from the central region. The inwardly-extending portions alternate with the outwardly-extending portions around the base of the closure device. When the closure device assumes the resting state, the base of the closure device thus may be similar to the shape of an asterisk or a flower having a plurality of petals. The tissue anchors are coupled to the base of the closure device such that when the closure device assumes the resting state, a first set of two or more of the tissue anchors are coupled to respective ones of the inwardly-extending portions, and a second set of two or more of the tissue anchors are coupled to respective ones of the outwardly-extending portions. Typically, an area of the opening when the base assumes the resting state is less than 80% of the area of the opening when the base of the closure device assumes the stretched state. For example, the area of the opening when the base assumes the resting state is 55% of the area of the opening when the base of the closure device assumes the stretched state.

Typically, the base of the closure device is continuous. Typically, the base of the closure device is configured such that, as the base of the closure device transitions from the stretched state toward the resting state, all of the anchors move in generally radial directions, and do not move in generally circumferential directions. Such radial motion is less likely to tear or otherwise damage the tissue of the myocardium than is circumferential motion. The hearts of older patients, upon whom cardiac procedures are most commonly performed, are particularly vulnerable to such tearing.

The base of the closure device is typically configured such that, as the closure device transitions from the stretched state toward the resting state, the anchors of the first, inner set move a greater distance than the anchors of the second, outer set. Movement by these two distances has the effect of applying two strengths of closure on the heart muscle: an inner, greater level of closure, surrounded by an outer, lesser level of closure. Together, the two levels of closure together tightly close the passage made through the myocardium, while minimizing the risk of damaging heart tissue.

In some applications of the present invention, a delivery tool and procedure are provided for performing a transapical surgical procedure. For some applications, the procedure uses the closure device described hereinabove, while for other applications, other closure techniques are used.

For some applications, the delivery tool comprises at a closure-device holder at a distal portion of the delivery tool. The holder is configured to hold the closure device and is moveable proximally and distally along a longitudinal axis of the delivery tool. The delivery tool comprises one or more closure-device-shape-adjusting elements coupled to the closure-device holder. The one or more closure-device-shape-adjusting elements are each shaped so as to define a coupling for removably coupling the closure device to the one or more closure-device-shape-adjusting elements. The one or more closure-device-shape-adjusting elements are configured to adjust a shape of the closure device by being moveable radially inwardly toward the longitudinal axis of the delivery tool to transition the closure device from the stretched state toward the resting state.

For some applications of the present invention, the anchors each comprise a pointed tip at a distal end of the tissue anchor, a barbed surface proximal to the pointed tip and configured to prevent proximal movement of the tissue anchor following penetration of the tissue anchor in the tissue of the patient, and a body portion extending between the pointed tip and the barbed surface. Each anchor defines a tissue-cutting portion extending directly from the pointed tip. Typically, the tissue-cutting portion extends 20-30% of a length of the body portion of the tissue anchor and is configured to cut the tissue of the patient. Each anchor defines a tissue-expanding portion disposed proximal to the tissue-cutting portion. Typically, the tissue-expanding portion extends 70-80% of the body portion of the tissue anchor and is configured to expand but not cut the tissue of the patient.

The above-mentioned tools and procedures advantageously enable minimally invasive access to the ventricles, and, via the ventricles, to the atria, aorta, and pulmonary blood vessels. The procedures generally do not require spreading of the patient's ribs, general anesthesia, mechanical ventilation, or the performance of an open thoracotomy. The procedures thus generally reduce patient pain during and after surgery, and minimize the likelihood of complications.

There is therefore provided, in accordance with some applications of the present invention, apparatus, including:
an implantable cardiac closure device having a stretched state and a resting state, the closure device being configured for facilitating closure of a hole made through tissue of a myocardial wall of a patient; and
a delivery tool including:
   a closure-device holder, at a distal portion of the delivery tool, being configured to hold the closure device, the closure-device holder being moveable proximally and distally along a longitudinal axis of the delivery tool; and
   one or more closure-device-shape-adjusting elements coupled to the closure-device holder, the one or more closure-device-shape-adjusting elements each being shaped so as to define a coupling for removably coupling the closure device to the one or more closure-device-shape-adjusting elements, the one or more closure-device-shape-adjusting elements being configured to adjust a shape of the closure device by being moveable radially inwardly toward the longitudinal axis of the delivery tool to transition the closure device from the stretched state toward the resting state.

In some applications of the present invention, the one or more closure-device-shape-adjusting elements are configured to further adjust the shape of the closure device by being moveable radially outwardly away from the longitudinal axis of the delivery tool to transition the closure device from the resting state toward the stretched state.

In some applications of the present invention, the distal portion of the delivery tool includes a casing within which the closure-device holder moves proximally and distally, the casing having a distal end which remains stationary during movement of the closure-device holder proximally and distally along the longitudinal axis of the delivery tool.

In some applications of the present invention, when the holder is moved proximally, the closure device is disposed entirely within the casing.

In some applications of the present invention, the apparatus includes a cap reversibly coupled to the casing, and the cap protects the closure device when the closure device and the closure-device holder are disposed at a distal portion of the casing.

In some applications of the present invention, when the closure device is disposed at a distal portion of the casing, tissue anchors of the closure device extend beyond the distal end of the casing.

In some applications of the present invention, the casing is shaped so as to define one or more windows, and the cap is shaped so as to define one or more radially-moveable legs, each one of the radially-moveable legs is shaped so as to define a protrusions which protrude into a respective one of the one or more windows when the cap is coupled to the casing and when the holder is disposed at the distal portion of the casing.

In some applications of the present invention, when the holder is moved proximally within the casing, the holder contacts and pushes against the one or more protrusions and pushes radially the one or more protrusions in order to decouple the one or more protrusions from the respective one or more windows in order to facilitate decoupling of the cap from the delivery tool.

In some applications of the present invention, the apparatus includes a control element at a proximal portion of the delivery tool, the control element being operatively associated with the one or more closure-device-shape-adjusting elements in order to gradually move the one or more closure-device-shape-adjusting elements radially in order to gradually transition the closure device between the stretched and resting states.

In some applications of the present invention, the control element includes a user interface including a knob that is (a) gradually rotatable in a first rotational direction to move the one or more closure-device-shape-adjusting elements radially outward in order to gradually transition the closure device from the resting state toward the stretched state, and (b) gradually rotatable in a second rotational direction to move the one or more closure-device-shape-adjusting elements radially inward in order to gradually transition the closure device from the stretched state toward the resting state.

In some applications of the present invention, the control element includes a motor that is (a) automatically actuatable to move the one or more closure-device-shape-adjusting elements radially outward in order to gradually transition the closure device from the resting state toward the stretched state, and (b) automatically actuatable to move the one or more closure-device-shape-adjusting elements radially inward in order to gradually transition the closure device from the stretched state toward the resting state.

In some applications of the present invention, the closure-device holder has a surface against which the closure device is positioned.

In some applications of the present invention, the one or more closure-device-shape-adjusting elements are moveable radially along the surface of the closure-device holder.

In some applications of the present invention, the surface of the closure-device holder includes a planar surface having a plane disposed perpendicularly to a longitudinal axis of the delivery tool.

In some applications of the present invention, the closure-device holder is shaped so as to define a rim at a perimeter of the distal end, the rim being shaped to provide one or more securing tabs which project radially toward a center of the delivery tool, each one of the one or more securing tabs being configured to maintain coupling of the closure device to the delivery tool when a respective portion of the closure device is disposed in a space defined by (a) a portion of the rim of the closure-device holder, (b) a portion of the surface of the closure-device holder, and (c) the one or more securing tabs.

In some applications of the present invention, the one or more closure-device-shape-adjusting elements move the respective portion of the closure device radially inwardly to release the respective portion of the closure device from the space and thereby to facilitate initial decoupling of the closure device from the closure-device holder.

In some applications of the present invention, the delivery tool further includes:
a spring operatively associated with the closure-device holder and compressible proximally along the longitudinal axis of the delivery tool into a compressed state to draw proximally the closure-device holder into a loaded state; and
a trigger operatively associated with the spring and moveable to facilitate releasing of the spring from the compressed state and firing of the closure-device holder distally.

In some applications of the present invention, the apparatus includes a lock coupled to the trigger, the lock being configured, in a locked state, to lock the trigger and maintain the spring in the compressed state and the closure-device holder in the loaded state, the lock being moveable into an unlocked state to release the trigger.

In some applications of the present invention:
the closure device is coupled to the delivery tool in the resting state,
the delivery tool is configured to:
   (a) transition the closure device into the stretched state,
   (b) subsequently, deliver the closure device into the tissue of the myocardial wall surrounding the hole made through the tissue, while the closure device is in the stretched state, and
   (c) subsequently, transition the closure device from the stretched state toward the resting state to facilitate closure of the hole in the tissue.

In some applications of the present invention, the closure device includes a superelastic material.

There is additionally provided, in accordance with some applications of the present invention, apparatus, including:
an implantable cardiac closure device having a stretched state and a resting state, the closure device being configured for facilitating closure of a hole made through tissue of a myocardial wall of a patient; and
a delivery tool including:
   a closure-device holder, at a distal portion of the delivery tool, being configured to hold the closure device, the closure-device holder being moveable proximally and distally along a longitudinal axis of the delivery tool;
   a casing at the distal portion of the delivery tool, the closure-device holder being moveable proximally and distally within the casing, the casing having a distal end which remains stationary during movement of the closure-device holder proximally and distally along the longitudinal axis of the delivery tool; and
   a cap reversibly coupled to the casing, the cap being configured to protect the closure device when the closure device and the closure-device holder are disposed at a distal portion of the casing,
   wherein the cap is decoupled from the delivery tool responsively to proximal movement of the closure-device holder.

There is further provided, in accordance with some applications of the present invention, tissue anchor for implantation in tissue of a patient, the tissue anchor including:
a pointed tip at a distal end of the tissue anchor;
a barbed surface proximal to the pointed tip and configured to prevent proximal movement of the tissue anchor following penetration of the tissue anchor in the tissue of the patient;
a body portion extending between the pointed tip and the barbed surface;
a tissue-cutting portion extending directly proximal from the pointed tip, the tissue-cutting portion extending 20-30% of a length of the body portion of the tissue anchor and configured to cut the tissue of the patient; and
a tissue-expanding portion disposed proximal to the tissue-cutting portion, the tissue-expanding portion extending 70-80% of the length of the body portion of the tissue anchor and configured to expand but not cut the tissue of the patient.

In some applications of the present invention, the tissue-cutting portion has a height of 0.3-0.7 mm measured along a central longitudinal axis of the tissue anchor.

In some applications of the present invention, the tissue-cutting portion has a height of 0.5 mm measured along a central longitudinal axis of the tissue anchor.

In some applications of the present invention, the tissue-expanding portion has a height of 1.0-2.0 mm measured along a central longitudinal axis of the tissue anchor.

In some applications of the present invention, the tissue-expanding portion has a height of 1.7 mm measured along a central longitudinal axis of the tissue anchor.

In some applications of the present invention, the tissue-cutting portion has a cutting angle of 40-60 degrees.

In some applications of the present invention, the tissue-cutting portion has a cutting angle of 54 degrees.

In some applications of the present invention, the anchor has a width of 0.3-1.5 mm.

In some applications of the present invention, the tissue-cutting portion includes at least one cutting edge that slopes 40-50 degrees from the pointed tip.

In some applications of the present invention, the tissue-cutting portion includes at least one cutting edge that slopes 45 degrees from the pointed tip.

In some applications of the present invention, the tissue-cutting portion includes two cutting edges that are disposed symmetrically with respect to a central longitudinal axis of the tissue anchor.

In some applications of the present invention:
the at least one cutting edge includes a first cutting edge,
the body portion includes a first flat surface that extends from a first edge of the barbed surface and distally toward a first converging point at the first flat surface,
the first converging point is disposed distal to the first edge of the barbed surface,
the first converging point defines an end of the first cutting edge,
the first cutting edge extends angularly between the first converging point and the pointed distal tip, and
the first flat surface has a first-flat-surface plane that is disposed in parallel with a central longitudinal axis of the tissue anchor at the pointed distal tip.

In some applications of the present invention:
the at least one cutting edge includes a second cutting edge,
the body portion includes a second flat surface that extends from a second edge of the barbed surface and distally toward a second converging point at the first second surface,
the second converging point is disposed distal to the second edge of the barbed surface,
the second converging point defines an end of the second cutting edge,
the second cutting edge extends angularly between the second converging point and the pointed tip, and
the second flat surface has a second-flat-surface plane that is disposed in parallel with a central longitudinal axis of the tissue anchor at the pointed distal tip.

In some applications of the present invention, the first cutting edge has a length of 0.1-2.0 mm.

In some applications of the present invention, the tissue-cutting portion is shaped so as to define a pyramid shape of the tissue anchor from the pointed tip toward the converging point.

In some applications of the present invention, the tissue-cutting portion is shaped so as to define a diamond shape of the tissue anchor from the pointed tip to the converging point.

In some applications of the present invention, the second cutting edge has a length of 0.1-2.0 mm.

In some applications of the present invention, the body portion has a width of 0.3-1.5 mm at the barbed surface, the width being measured between the first and second edges of the barbed surface.

In some applications of the present invention, the first and second flat surfaces are parallel.

There is yet further provided, in accordance with some applications of the present invention, a method for grinding a tissue anchor, including:
providing a tissue anchor having at least a base portion and an anchoring portion, the anchoring portion being configured to be embedded within tissue of a patient, the anchoring portion having four surfaces; and
creating four additional surfaces of the tissue anchor at the anchoring portion by:
   creating a fifth surface of the tissue anchor at the anchoring portion by:
      rotating the tissue anchor about a central longitudinal axis 40-50 degrees in a first rotational direction;
      tilting the tissue anchor 15-25 degrees from the central longitudinal axis in a first tilting direction; and
      subsequently to the tilting of the tissue anchor in the first tilting direction, grinding the tissue anchor for a first grinding;
   subsequently to the grinding of the anchor for the first grinding, creating a sixth surface of the tissue anchor at the anchoring portion by:
      tilting the tissue anchor back to an upright position;
      subsequently, tilting the tissue anchor to the upright position, tilting the tissue anchor 15-25 degrees from the central longitudinal axis in a second tilting direction opposite the first tilting direction; and
      subsequently to the tilting the tissue anchor in the second tilting direction, grinding the tissue anchor for a second grinding;
   subsequently to the grinding of the anchor for the second grinding, creating a seventh surface of the tissue anchor at the anchoring portion by:
      rotating the tissue anchor 80-100 degrees about the central longitudinal axis in a second rotational direction;
      tilting the tissue anchor 15-25 degrees from the central longitudinal axis in a third tilting direction; and
      subsequently to the tilting of the tissue anchor in the third tilting direction, grinding the tissue anchor for a third grinding; and
   subsequently to the grinding of the anchor for the third grinding, creating an eighth surface of the tissue anchor at the anchoring portion by:
      tilting the tissue anchor back to the upright position;
      subsequently to the tilting the tissue anchor to the upright position, tilting the tissue anchor 15-25 degrees from the central longitudinal axis in a fourth tilting direction opposite the third tilting direction; and
      subsequently to the tilting the tissue anchor in the fourth tilting direction, grinding the tissue anchor for a fourth grinding.

There is also provided, in accordance with some applications of the present invention, an implantable cardiac closure device configured for facilitating closure of a hole made through tissue of a myocardial wall of a patient, the closure device including:
a base having at least one projection having a shape having two or more projection interfaces; and
at least one tissue anchor having at least a base portion and an anchoring portion, the anchoring portion being configured to be embedded within tissue of a patient, the base portion being shaped so as to define a space corresponding to the shape of the at least one projection of the base, the base portion having two or more base-portion interfaces engaging the two or more projections interfaces of the projection of the base,
wherein each one of the two or more projection interfaces is welded to a respective one of the two or more base-portion interfaces.

There is further provided, in accordance with some applications of the present invention, a method, including:
providing an implantable cardiac closure device and a delivery tool, the delivery tool including:
   a closure-device holder at a distal portion of the delivery tool, the closure-device holder being configured to hold the closure device, the closure-device holder being moveable proximally and distally along a longitudinal axis of the delivery tool; and
   one or more closure-device-shape-adjusting elements coupled to the closure-device holder, the one or more closure-device-shape-adjusting elements each being shaped so as to define a coupling for removably coupling the closure device to the one or more closure-device-shape-adjusting elements; and
using the delivery tool:
   implanting the closure device in a body of a patient in tissue of a myocardial wall surrounding a hole made through the tissue; and
   facilitating closing of the hole by adjusting a shape of the closure device, by moving the one or more closure-device-shape-adjusting elements radially inwardly toward the longitudinal axis of the delivery tool, thereby transitioning the closure device from a stretched state toward a resting state.

In some applications of the present invention, the method further includes, subsequently to the implanting of the closure device, and prior to the facilitating of the closing of the opening in the tissue, performing replacement of a native cardiac valve of the patient by advancing a valve-replacement tool through the closure device, the valve-replacement tool being configured for replacing the native cardiac valve.

In some applications of the present invention, the method further includes, subsequently to the implanting of the closure device, and prior to the facilitating of the closing of the opening in the tissue, performing repair of cardiac tissue of the patient by advancing a cardiac-repair tool through the closure device, the cardiac-repair tool being configured for repairing the cardiac tissue.

In some applications of the present invention, performing the repair includes repairing a cardiac valve of the patient.

In some applications of the present invention, the method further includes, prior to the implanting of the closure device, moving the closure-device holder proximally along the longitudinal axis of the delivery tool to draw proximally the closure-device holder and the closure device.

In some applications of the present invention, moving the closure-device holder proximally along the longitudinal axis of the delivery tool includes moving the closure-device holder proximally with respect to a distal end of the delivery tool.

In some applications of the present invention, subsequently to the moving of the closure-device holder proximally, advancing the distal portion of the delivery tool into the body of the patient until the distal end of the delivery tool contacts the tissue surrounding the hole.

In some applications of the present invention, moving the closure-device holder proximally along the longitudinal axis of the delivery tool includes loading the closure-device holder into a loaded state by compressing a spring of the delivery tool responsively to moving the closure-device holder proximally.

In some applications of the present invention, the method further includes, firing the closure-device holder distally by, using a trigger of the delivery tool, (a) releasing the spring from the compressed state, and (b) facilitating the firing of the closure-device holder distally.

In some applications of the present invention, the method further includes, prior to the firing, locking the trigger and by the locking, maintaining the spring in the compressed state and the closure-device holder in the loaded state, and subsequently, unlocking the trigger in order to facilitate the firing.

In some applications of the present invention, the closure-device holder has a surface against which the closure device is positioned and moving the one or more closure-device-shape-adjusting elements includes moving the one or more closure-device-shape-adjusting elements radially along the surface of the closure-device holder.

In some applications of the present invention, the surface of the closure-device holder includes a planar surface having a plane disposed perpendicularly to a longitudinal axis of the delivery tool.

In some applications of the present invention:
the closure-device holder is shaped so as to define a rim at a perimeter of the distal end, the rim being shaped to provide one or more securing tabs which project radially toward a center of the delivery tool, and
gradually moving the closure device into the resting state includes releasing the closure device from a space defined by (a) a portion of the rim of the closure-device holder, (b) a portion of the surface of the closure-device holder, and (c) the one or more securing tabs.

In some applications of the present invention:
the delivery tool includes a control element including a user interface, the control element being operatively associated with the one or more closure-device-shape-adjusting elements,
moving the one or more closure-device-shape-adjusting elements radially inwardly includes gradually moving the one or more closure-device-shape-adjusting elements by actuating the control element, and by the gradually moving the one or more closure-device-shape-adjusting elements radially inwardly, gradually transitioning the closure device toward the resting state.

In some applications of the present invention:
the control element includes a knob,
actuating the control element includes rotating the knob gradually in a first rotational direction, and
moving the one or more closure-device-shape-adjusting elements radially inwardly includes moving the one or more closure-device-shape-adjusting elements radially inwardly responsively to the rotating the knob gradually in the first rotational direction.

In some applications of the present invention, prior to the implanting of the closure device, expanding the closure device into the stretched state by moving the one or more closure-device-shape-adjusting elements radially outwardly from the longitudinal axis of the delivery tool.

In some applications of the present invention:
the delivery tool includes a control element including a user interface, the control element being operatively associated with the one or more closure-device-shape-adjusting elements,
moving the one or more closure-device-shape-adjusting elements radially outwardly includes gradually moving the one or more closure-device-shape-adjusting elements by actuating the control element, and
by the gradually moving the one or more closure-device-shape-adjusting elements radially outwardly, gradually transitioning the closure device toward the stretched state.

In some applications of the present invention:
the control element includes a knob,
actuating the control element includes rotating the knob gradually in a second rotational direction,
moving the one or more closure-device-shape-adjusting elements radially outwardly includes moving the one or more closure-device-shape-adjusting elements radially outwardly responsively to the rotating the knob gradually in the second rotational direction.

There is also provided, in accordance with some applications of the present invention, a method, including:
providing an implantable cardiac closure device and a delivery tool, the delivery tool including a closure-device holder at a distal portion of the delivery tool, the closure-device holder being configured to hold the closure device, the closure-device holder being moveable proximally and distally along a longitudinal axis of the delivery tool;
using the delivery tool, expanding the closure device into a stretched state;
subsequently to the expanding, moving the closure-device holder proximally along the longitudinal axis of the delivery tool to draw proximally the closure-device holder and the closure device;
subsequently to the moving of the closure-device holder proximally, advancing the distal portion of the delivery tool into a body of the patient until the distal end of the delivery tool contacts tissue surrounding a hole made through tissue of a myocardial wall of the patient;
using the delivery tool, firing the closure-device holder distally while the closure device is in the stretched state, and by the firing, implanting the closure device in the tissue of the myocardial wall surrounding the hole made through the tissue, by facilitating penetrating of one or more barbs of the closure device into the tissue surrounding the hole; and
subsequently to the firing, facilitating closing of the hole in the tissue by actuating a control element at a proximal portion of the delivery tool, thereby gradually transitioning the closure device from the stretched state toward a resting state.

In some applications of the present invention, the method further includes, subsequently to the firing of the closure-device holder distally and prior to the facilitating of the closing of the opening in the tissue, performing replacement of a native cardiac valve of the patient by advancing a valve-replacement tool through the closure device, the valve-replacement tool being configured for replacing the native cardiac valve.

In some applications of the present invention, providing the delivery tool includes providing a delivery tool shaped so as to define a casing at the distal portion of the delivery tool, the casing within which the closure-device holder moves proximally and distally, the casing having a distal end which remains stationary during movement of the closure-device holder proximally and distally along the longitudinal axis of the delivery tool.

In some applications of the present invention, moving the closure-device holder proximally includes including moving the closure-device holder proximally within the casing toward a proximal position, and moving of the closure-device holder includes moving the closure device to be disposed entirely within the casing.

In some applications of the present invention, the method further includes, protecting the closure device by a cap reversibly coupled to the casing.

In some applications of the present invention, protecting the closure device by a cap reversibly coupled to the casing includes protecting the closure device by the cap when the closure device is disposed at a distal portion of the casing.

In some applications of the present invention:
the casing is shaped so as to define one or more windows,
the cap is shaped so as to define one or more radially-moveable legs, each one of the radially-moveable legs is shaped so as to define a protrusions which protrude into a respective one of the one or more windows when the cap is coupled to the casing and when the holder is disposed at the distal portion of the casing, and
moving the closure-device holder proximally includes moving the closure-device holder within the casing, and by the moving, facilitating pushing of the closure-device holder against the one or more protrusions, and thereby facilitating pushing radially the one or more protrusions and thereby facilitating decoupling of the cap from the delivery tool by facilitating decoupling of the one or more protrusions from the respective one or more windows.

In some applications of the present invention, facilitating decoupling of the cap includes facilitating decoupling of the cap prior to the advancing the distal portion of the delivery tool into the body of the patient.

In some applications of the present invention, the method further includes, subsequently to the firing of the closure-device holder distally and prior to the facilitating of the closing of the opening in the tissue, performing repair of cardiac tissue of the patient by advancing a cardiac-repair tool through the closure device, the cardiac-repair tool being configured for repairing the cardiac tissue.

In some applications of the present invention, performing the repair includes repairing a cardiac valve of the patient.

In some applications of the present invention, the closure-device holder has a surface against which the closure device is positioned.

In some applications of the present invention, the surface of the closure-device holder includes a planar surface having a plane disposed perpendicularly to a longitudinal axis of the delivery tool.

In some applications of the present invention:
the closure-device holder is shaped so as to define a rim at a perimeter of the distal end, the rim being shaped to provide one or more securing tabs which project radially toward a center of the delivery tool, and
gradually transitioning the closure device from the stretched state toward the resting state includes releasing the closure device from a space defined by (a) a portion of the rim of the closure-device holder, (b) a portion of the surface of the closure-device holder, and (c) the one or more securing tabs.

In some applications of the present invention:
the closure device is removably coupled to one or more closure-device-shape-adjusting elements coupled to the closure-device holder,
each one of the one or more closure-device-shape-adjusting elements is shaped so as to define a coupling for removably coupling the closure device to the respective one or more closure-device-shape-adjusting elements, and
expanding the closure device into the stretched state includes moving the one or more closure-device-shape-adjusting elements radially outwardly from the longitudinal axis of the delivery tool and along the surface of the closure-device holder.

81. The method according to claim 80:
the control element provides a user interface and is operatively associated with the one or more closure-device-shape-adjusting elements,
moving the one or more closure-device-shape-adjusting elements radially outwardly includes gradually moving the one or more closure-device-shape-adjusting elements by actuating the control element, and
by the gradually moving the one or more closure-device-shape-adjusting elements radially outwardly, gradually transitioning the closure device into the stretched state.

In some applications of the present invention:
the control element includes a knob,
actuating the control element includes rotating the knob gradually in a first rotational direction, and
moving the one or more closure-device-shape-adjusting elements radially outwardly includes moving the one or more closure-device-shape-adjusting elements radially outwardly responsively to the rotating the knob gradually in the first rotational direction.

In some applications of the present invention:
the closure device is removably coupled to one or more closure-device-shape-adjusting elements coupled to the closure-device holder,
each one of the one or more closure-device-shape-adjusting elements is shaped so as to define a coupling for removably coupling the closure device to the respective one or more closure-device-shape-adjusting elements, and
gradually transitioning the closure device from the stretched state toward the resting state includes moving the one or more closure-device-shape-adjusting elements radially inwardly from the longitudinal axis of the delivery tool and along the surface of the closure-device holder.

In some applications of the present invention:
the control element provides a user interface and is operatively associated with the one or more closure-device-shape-adjusting elements,
moving the one or more closure-device-shape-adjusting elements radially inwardly includes gradually moving the one or more closure-device-shape-adjusting elements by actuating the control element, and
by the gradually moving of the one or more closure-device-shape-adjusting elements radially inwardly, gradually transitioning the closure device into the resting state.

In some applications of the present invention:
the control element includes a knob,
actuating the control element includes rotating the knob gradually in a second rotational direction, and
moving the one or more closure-device-shape-adjusting elements radially inwardly includes moving the one or more closure-device-shape-adjusting elements radially inwardly responsively to rotating the knob gradually in the second rotational direction.

In some applications of the present invention, moving the closure-device holder proximally along the longitudinal axis of the delivery tool includes loading the closure-device holder into a loaded state by compressing a spring of the delivery tool responsively to moving the closure-device holder proximally.

In some applications of the present invention, firing the closure-device holder distally includes, using a trigger of the delivery tool, (a) releasing the spring from the compressed state, and (b) facilitating the firing of the closure-device holder distally.

In some applications of the present invention, the method further includes, prior to the firing, locking the trigger and by the locking, maintaining the spring in the compressed state and the closure-device holder in the loaded state, and subsequently, unlocking the trigger in order to facilitate the firing.

The present invention will be more fully understood from the following detailed description of embodiments thereof, taken together with the drawings, in which:

### BRIEF DESCRIPTION OF THE DRAWINGS

FIG. 1 is a schematic illustration of a closure device in a resting state, respectively, in accordance with some applications of the present invention;

FIGS. 2A-B are schematic illustrations of steps for grinding a tissue anchor of the closure device of FIG. 1, in accordance with some applications of the present invention;

FIG. 3 is a schematic illustration of a delivery tool for delivering and implanting the closure device of FIG. 1, in accordance with some applications of the present invention;

FIGS. 4A-F are schematic illustrations of operation of the delivery tool to transition the closure device between the resting and stretched states, in accordance with some applications of the present invention;

FIG. 5 is a schematic illustration of preparation of the delivery tool prior to introduction into a body of a patient, in accordance with some applications of the present invention;

FIGS. 6-9 are schematic illustrations of the delivery tool being used to facilitate performing of a transapical surgical procedure and closure of a hole made in myocardial tissue of the patient; and

FIG. 10 is a schematic illustration of a delivery tool for delivering and implanting the closure device of FIG. 1, in accordance with some other applications of the present invention.

### DETAILED DESCRIPTION OF APPLICATIONS

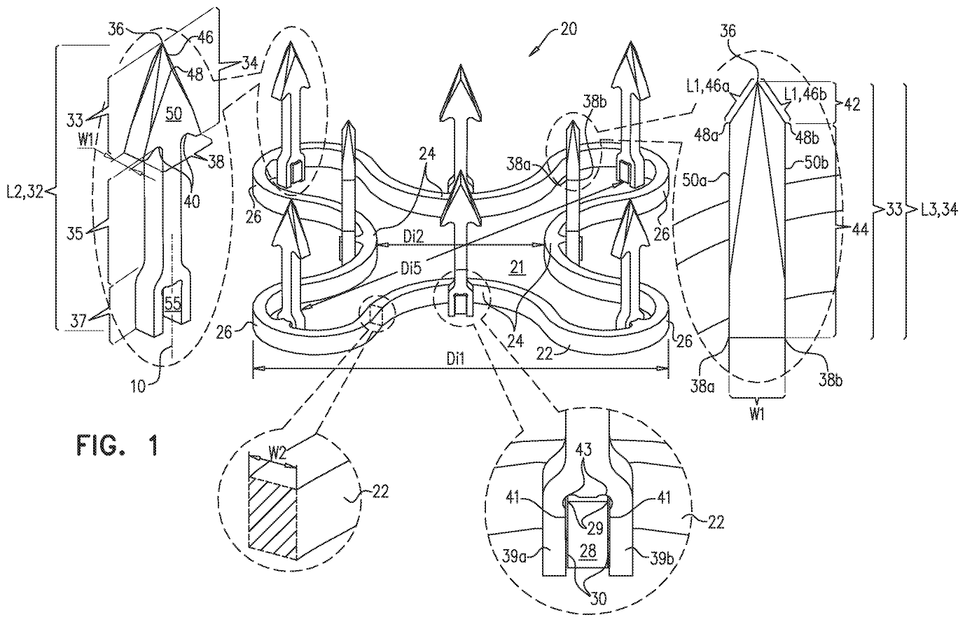

FIG. 1 is a schematic illustration of a closure device 20 comprising a base 22 and a plurality of tissue anchors 32, in accordance with some applications of the present invention. Typically, closure device 20 comprises an implant. Closure device 20 comprises a superelastic materiel (e.g., nitinol) or an elastic metal, such as elastic stainless steel, and has a stretched state and a resting state. For some applications of the present invention, closure device 20 comprises a shape-memory material. FIG. 1 shows closure device 20 in the resting state. Closure device 20 is configured for facilitating closure of a hole made through tissue of a myocardial wall of a patient, as is described hereinbelow.

Base 22 is shaped so as to define a continuous structure having an opening 21 therethrough. Typically, base 22 is non-circular. Base 22 is typically clover-shaped, as shown. For some applications of the present invention, base 22 is flower-shaped. For some applications, base 22 is shaped so as to define at least one petal shape, e.g., four petal shapes, as shown. For some applications, base 22 is flat in the stretched, partially stretched, and resting states of device 20, i.e., would define exactly one plane if the material of base 22 were to be conceptualized as a line without thickness; if placed on a flat surface, the base would touch the surface at all point along the entire base. Alternatively, base 22 is generally, but not entirely, flat.

Typically, base 22 comprises a superelastic materiel (e.g., nitinol) and has a width W2 of 0.5-1.0 mm (e.g., 0.8 mm).

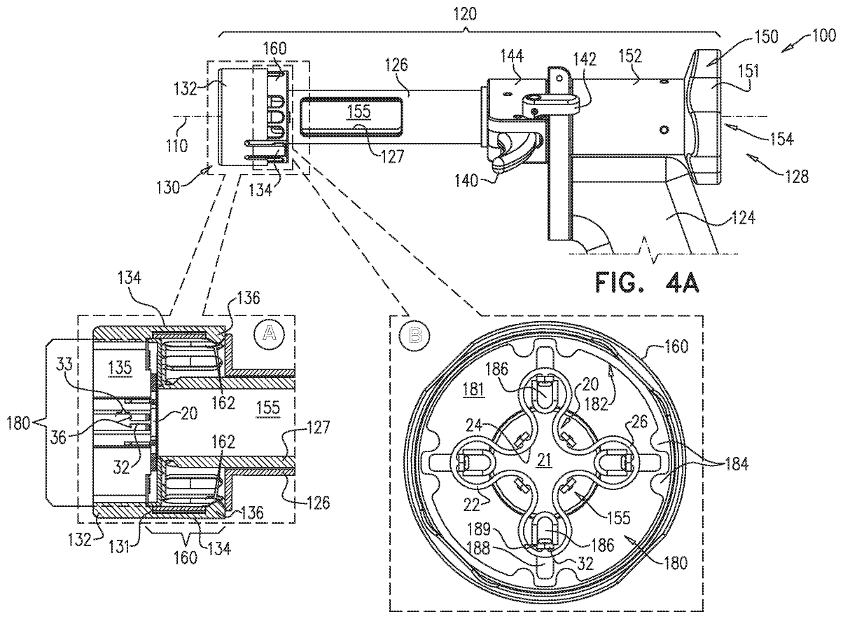

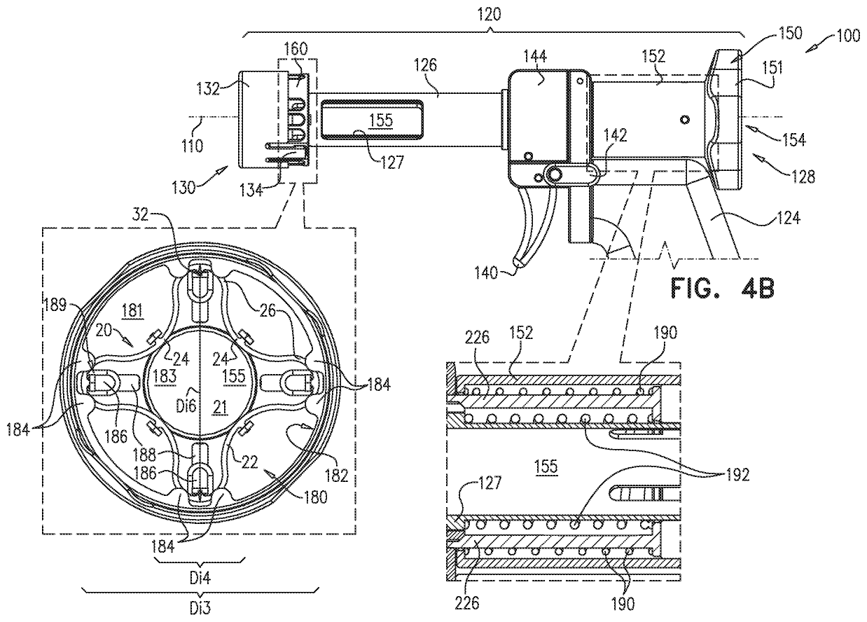

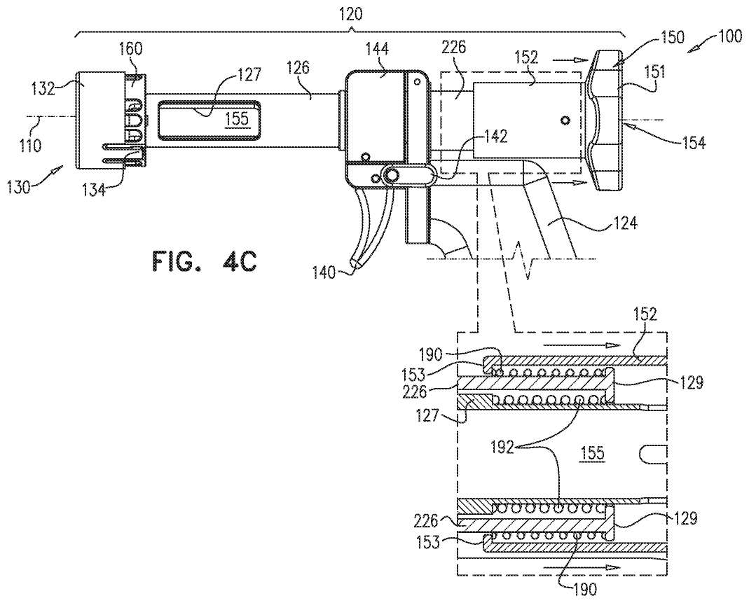

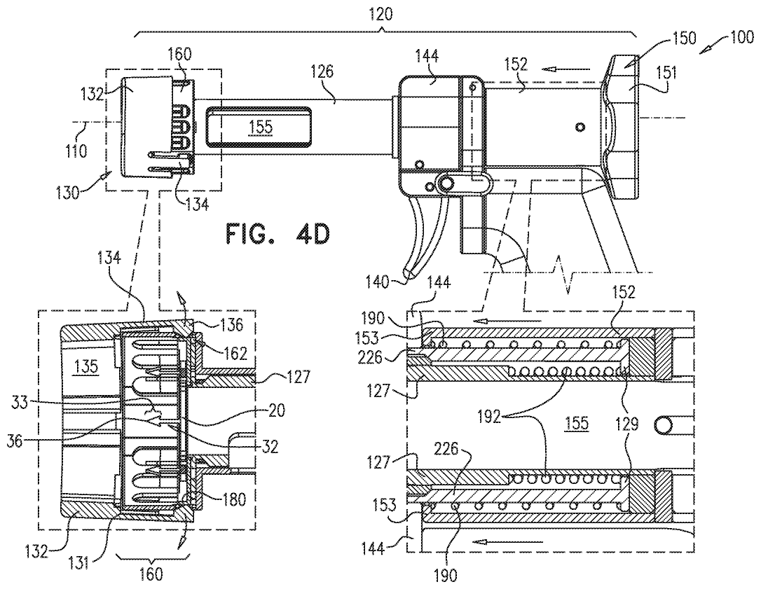

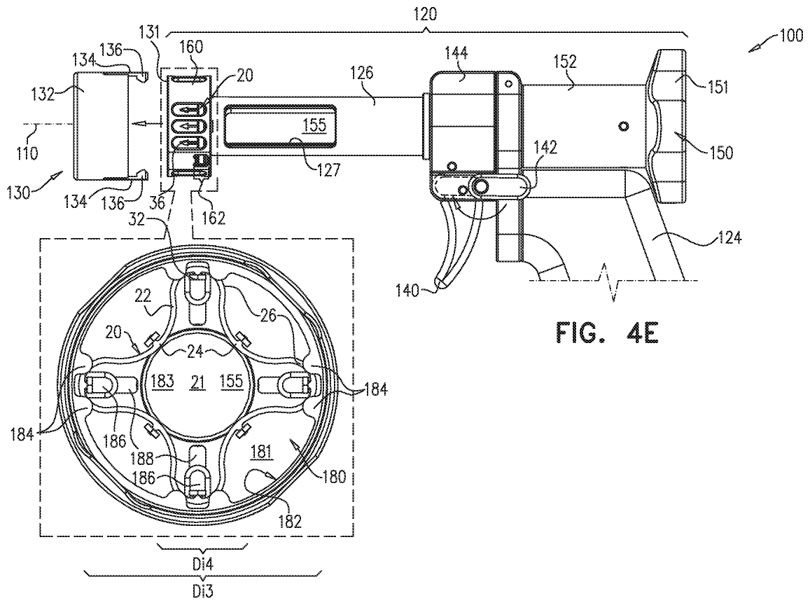

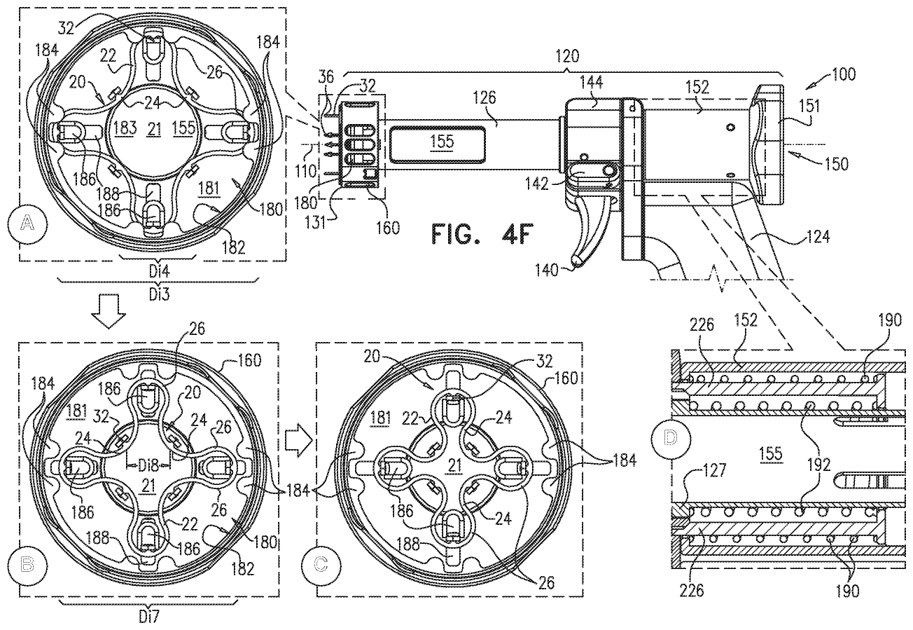

As shown in the resting state of closure device 20 in FIG. 1, opening 21 has a resting-state area that is between 20% and 80% of opening 21 when closure device 20 is in the stretched state, as is shown in FIG. 4B hereinbelow. For example, opening 21 has a resting-state area that is less than 80% of the area of opening 21 in the stretched state, such as less than 60% or less than 40% of the area of opening 21 in the stretched state. For example, the resting-state area may be between 10 mm2 and 565 mm2, such as between 15 mm2 and 393 mm2, e.g., 127 mm2.

Typically, in the resting state and in the stretched state of closure device 20, base 22 is shaped so as to define: (a) two or more (e.g., 4, as shown) inwardly-extending portions 24, which extend toward a central region of base 22, and (b) two or more (e.g., 4, as shown) outwardly-extending portions 26, which extend away from the central region of base 22. Inwardly-extending portions 24 alternate with outwardly-extending portions 26 around base 22. For some applications, base 22 is shaped so as to define between two and ten inwardly-extending portions 24 and between two and ten outwardly-extending portions 26, such as exactly two, exactly three, or exactly eight of each type of portion. For some applications, a greatest distance Di1 across closure device 20 in the resting state and including base 22, as shown in FIG. 1, is 10-20 mm, e.g., 18.9 mm, and a closest distance Di2 between two opposite inwardly-extending portions 24 in the resting state is 3-10 mm, e.g., 4 mm. Additionally, in the resting state, closure device 20 has a greatest distance Di5 across opening 21 of closure device 20, between any two opposite outwardly-extending portions 26 of 5-19 mm, e.g., 15 mm.

Closure device 20 further comprises four or more tissue anchors 32, e.g., eight, as shown. Each one of tissue anchors 32 is shaped so as to define a respective distal anchoring portion 33, a post 35, and a base portion 37. Each anchor 32 typically comprises a superelastic material (e.g., nitinol). Typically, anchoring portion 33, a post 35, and a base portion 37 are formed from a single piece. Each one of anchoring portions 33 is typically shaped so as to define a pointed tip 36 at a distal end of each anchor 32 and a generally pyramid-shaped portion extending proximally from pointed tip 36. For some applications, the portion extending proximally from pointed tip 36 is shaped so as to define a generally diamond shape. Anchors 32 are coupled to base 22 such that each anchor 32 defines an angle of between 75 and 115 degrees with a plane defined by opening 21 of device 20, such as between 85 and 95 degrees, e.g., 90 degrees. Typically, closure device 20 comprises between 6 and 20 anchors 32, such as exactly 8 or exactly 12 anchors. For some applications, the number of anchors equals the sum of the number of inwardly-extending portions 24 and the number of outwardly-extending portions 26. Alternatively, the number of anchors is less than or greater than the sum. Typically, each of anchors 32 has a length L2 of between 2 and 10 mm, such as between 5 and 6 mm (e.g., 8 mm), or between 1 and 6 mm, such as between 2 and 5 mm (e.g., 3 mm).

For some applications, at least a portion (such as all, as shown) of tissue anchors 32 are shaped to define respective barbed surfaces 38 at a proximal end of each one of anchoring portions 33 such that anchoring portions 33 define barbs. The barbs help couple the anchors to the muscle tissue of the myocardium, generally irreversibly. Barbed surfaces 38 are each shaped so as to define a plane that is perpendicular to a central longitudinal axis 10 of anchor 32. Barbed surface 38 is shaped so as to define one or more indented sections 40 which help couple the anchors to the muscle tissue as the muscle tissue closes around anchoring portion 33 and into indented sections 40. Barbed surface 38 is configured to prevent proximal movement of the tissue anchor following penetration of tissue anchor 32 in the tissue of the patient.

Each anchoring portion 33 has a body portion 34 that is disposed between pointed tip 36 and barbed surface 38. Anchoring portions 33 are each shaped so as to define first and second flat surfaces 50a and 50b, respectively, on either side of anchor 32 at body portion 34. Typically, first flat surface 50a has a plane that is disposed in parallel with central longitudinal axis 10 of anchor 32. Typically, second flat surface 50b has a plane that is disposed in parallel with central longitudinal axis 10 of anchor 32. Typically, first and second flat surfaces 50a and 50b are parallel. Each of first and second flat surfaces 50a and 50b has a respective converging point 48a and 48b that is proximal to pointed tip 36. Each one of flat surfaces 50a and 50b extends from a respective edge 38a and 38b of barbed surface 38 and toward a respective converging point 48a and 48b. That is, converging points 48a and 48b are disposed distal to edges 38a and 38b of barbed surface 38. Anchor 32 has a width W1 of 0.3-1.5 mm (e.g., 0.5 mm) at barbed surface 38. For some applications, width W1 is measured between first and second edges 38a and 38b of barbed surface 38 of body portion 34.

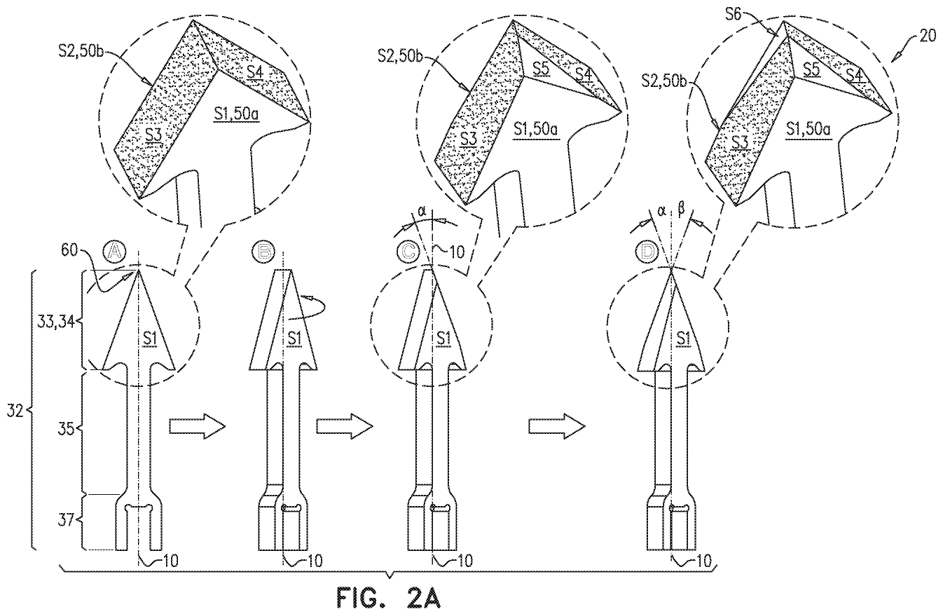

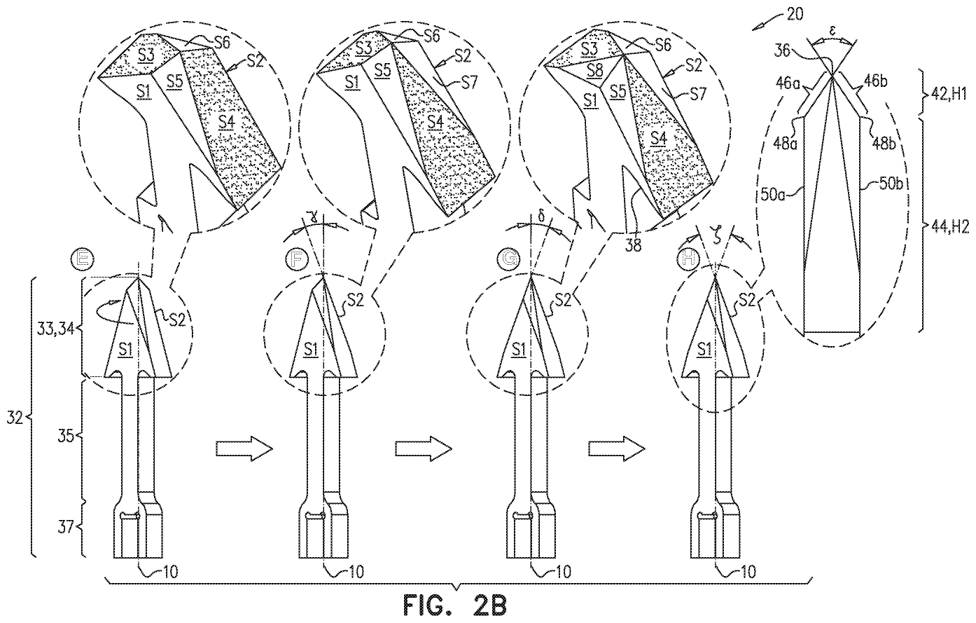

Each anchoring portion 33 of each anchor 32 is sharpened (as is described hereinbelow with reference to FIGS. 2A-B) such that it is shaped so as to define a tissue-cutting portion 42 extending directly from pointed tip 36. Typically, tissue-cutting portion 42 extends 20-30% of a length L3 of body portion 34 of each tissue anchor 32. Length L3 is typically between 1-5 mm. Each tissue-cutting portion 42 is configured to cut the tissue of the patient. Tissue-cutting portion 42 is shaped so as to define a pyramid shape of anchor 32 from pointed tip 36 toward converging points 48a and 48b. For some applications, tissue-cutting portion 42 is shaped so as to define a diamond shape of anchor 32 from pointed tip 36 toward converging points 48a and 48b. Anchoring portion 33 of each anchor 32 is shaped so as to define a tissue-expanding portion 44 disposed proximal to tissue-cutting portion 42. Each tissue-expanding portion 44 extends 70-80% of length L3 of body portion 34 of tissue anchor 32. Each tissue-expanding portion 44 is configured to expand but not cut the tissue of the patient.

Each tissue-cutting portion 42 comprises at least one (e.g., two) cutting edge. As shown, each tissue-cutting portion 42 comprises first and second cutting edge 46a and 46b. Each of cutting edges 46a and 46b slopes 40-50 degrees, e.g., 45 degrees, from pointed tip 36. Cutting edges 46a and 46b are disposed symmetrically with respect to central longitudinal axis 10 of anchor 32.

First cutting edge 46a extends angularly between first converging point 48a and distal pointed tip 36. First converging point 48a defines an end of first cutting edge 46a. Second cutting edge 46b extends angularly between second converging point 48b and distal pointed tip 36. Second converging point 48b defines an end of second cutting edge 46b. Each of first and second cutting edges has a length L1 of 0.1-2.0 mm, e.g., 0.5 mm.

Extending proximally from anchoring portion 33 is post 35, which is typically straight. Base portion 37 is disposed proximally to post 35 and comprises first and second legs 39a and 39b which surround a space 55 of base portion 37. Legs 39a and 39b facilitate coupling of anchor 32 to base 22 of closure device 20. Base 22 is shaped so as to define a plurality of projections 28 which project radially with respect to a center of opening 21 of device 20. For some applications, each projection 28 is shaped as a cube. For some applications, each projection 28 is shaped as a cuboid. For some applications, projections 28 coupled to inwardly-extending portions 24 of base 22 extend away from the center of opening 21 of device 20, and projections 28 coupled to outwardly-extending portions 26 of base 22 extend toward the center of opening 21 of device 20. Typically, the number of projections 28 of base 22 corresponds to the number of tissue anchors 32 of closure device 20. For some applications, projections 28 are flush with base 22 in the stretched, partially stretched, and resting states of device 20, i.e., would define exactly one plane if the material of base 22 were to be conceptualized as a line without thickness; if placed on a flat surface, base 22 would touch the surface at all point along the entire base, including projections 28.

Each projection 28 defines respective projection interfaces 30 which align with respective base-portion interfaces 41 of each of legs 39a and 39b of a respective base portion 37 of each anchor 32. That is, each base portion 37 is shaped so as to define a geometric receiving space for coupling of a respective anchor 32 to a respective projection 28 of base 22. Legs 39a and 39b fit around projection 28. Additionally, base portion 37 is shaped so as to define indentations 43 which are shaped so as to provide a space for movement of corners 29 of each projection 28 during the initial coupling of each anchor 32 to a respective projection 28 of base 22 during manufacture of closure device 20. That is, during manufacture, legs 39a and 39b of each anchor slide proximally along interfaces 30 of a respective projection 28 of base portion 37 until corners 29 of projection 28 move into and are released within indentations 43 of base portion 37. Thus, projection 28 fits within base portion 37. As such, for each anchor 32, closure device 20 provides a respective geometric coupling between (1) base portion 37, legs 39a and 39b and their respective interfaces 41, and indentations 43, and (2) projection 28 and its interfaces 30 and corners 29.

Once projection 28 fits geometrically within space 55 of anchor base portion 37, base portion 37 is fixedly coupled (e.g., welded) to projection 28 of base 22 of closure device 20. That is, space 55 corresponds to the geometric shape of projection 28 of base 22. That is, for each anchor 32, closure device 20 provides a respective geometric coupling as well as a mechanical coupling, typically, but not necessarily, by welding. Typically, interfaces 41 of base portion 37 of anchor 32 are welded to interfaces 30 of projection 28. The combination of (1) welding interfaces 41 and 30 providing a geometric locking (e.g., a fork shape) and (2) welding between respective interfaces 41 and 30 achieves minimal loads in the welding area. In order to provide strength to the superelastic material of closure device 20, the geometric locking typically absorbs the major external loads on anchors 32 and the welding functions to hold the anchor 32 in place with respect to base 22. Most of the welding is performed at the proximal portion of projection 28 which tends to assume minimal stress from the external loads.

FIGS. 2A-B are schematic illustrations of steps for grinding anchoring portion 33 of tissue anchor 32 of closure device 20 of FIG. 1, in accordance with some applications of the present invention. In stage A of FIG. 2A, anchor 32 is disposed in alignment symmetrically about a central longitudinal axis 10. As shown in stage A, anchoring portion 33 of anchor 32 has not yet been ground and defines four surfaces: (a) a first surface S1 which defines first flat surface 50a, (b) a second surface S2 which defines second flat surface 50b, (c) a third surface S3 that slopes from a distal-most end 60 of tissue anchor 32, and (d) a fourth surface S4 that slopes from distal-most end 60 of tissue anchor 32 symmetrically with respect to third surface S3. As described hereinabove, first and second surfaces S1 and S2 each define a respective a plane that is disposed in parallel with central longitudinal axis 10 of anchor 32 at pointed tip 36. Typically, first and second surfaces S1 and S2 are parallel and disposed opposite each other.

For some applications, surfaces S3 and S4 are blasted (e.g., sand blasting or glass bead blasting) in order to prevent surfaces S3 and S4 from cutting tissue.

Prior to grinding anchoring portion 33 for a first time, anchor 32 is rotated in a first rotational direction about its central longitudinal axis 10 about 40-50 degrees, e.g., typically 45 degrees, as is shown in stage B.

In stage C, after being rotated, anchor 32 is tilted at a first tilting angle α (alpha) in a first tilting direction from central longitudinal axis 10 toward a grinding surface of a grinding machine, which grinds a portion of anchoring portion 33. Typically, angle α (alpha) is 15-25 degrees (e.g., 20 degrees) from central longitudinal axis 10. Typically, angle α (alpha) defines a primary grind angle. After the first grinding, anchoring portion 33 defines a fifth surface S5.

Anchor 32 is then tilted back to an upright position as in stage B, and then is tilted in the a second tilting direction opposite direction the first tilting direction shown in stage C and toward the grinding surface of the grinding machine, such that anchor 32 is tilted at a second tilting angle β (beta) from central longitudinal axis 10 and opposite the first tilting angle α (alpha). Typically, angle β (beta) is 15-25 degrees (e.g., 20 degrees) from central longitudinal axis 10. Once tilted to angle β (beta), as shown in stage D, anchor 32 is ground for a second time in order to achieve a sixth surface S6 of anchoring portion 33.

Reference is now made to FIG. 2B. Subsequently to stage D in FIG. 2A, in stage E, anchor 32 is rotated 80-100 degrees (e.g., 90 degrees) about central longitudinal axis 10 in a second rotational direction opposite the first rotational direction.

In stage F, after being rotated, anchor 32 is tilted at a third tilting angle γ (gamma) in a third tilting direction from central longitudinal axis 10 toward the grinding surface of the grinding machine, and is then ground by the grinding surface for a third grinding. Typically, angle γ (gamma) is 15-25 degrees (e.g., 20 degrees) from central longitudinal axis 10. After the third grinding, anchoring portion 33 defines a seventh surface S7.

In stage G, anchor 32 is then tilted back to an upright position as in stage E, and then is tilted in a fourth tilting direction opposite direction from the third direction shown in stage F and toward the grinding surface of the grinding machine, such that anchor 32 is tilted at a fourth angle δ (delta) from central longitudinal axis 10 and opposite the third tilting angle γ (gamma). Typically, angle δ (delta) is 15-25 degrees (e.g., 20 degrees) from central longitudinal axis 10. Once tilted to angle δ (delta), as shown in stage G, anchor 32 is ground for a fourth time in order to achieve an eighth surface S8 of anchoring portion 33.

In stage H, anchor 32 is fully ground and defines eight surfaces S1-S8 of anchoring portion 33. Once ground, anchoring portion 33 defines body portion 34 between barbed surface 38 and pointed tip 36. Following the grinding, anchor 32 defines anchoring portion 33 having a pointed tip 36, first and second converging points 48a and 48b and cutting edges 46a and 46b. Cutting edge 46a is formed from the grinding of surfaces S5 and S8, and cutting edge 46b is formed from the grinding of surfaces S6 and S7. Typically, cutting edges 46a and 46b slope 40-50 degrees, e.g., 45 degrees, from pointed tip 36. Cutting edges 46a and 46b are sharp and are configured to cut tissue of the patient. Thus, following the grinding, anchoring portion 33 defines tissue-cutting portion 42 extending directly from pointed tip 36. Taken together, cutting edges 46a and 46b of tissue-cutting portion 42 of anchoring portion 33 define a cutting angle c (epsilon) that is 40-60 degrees, e.g., 54 degrees. For some applications of the present invention, angle c (epsilon) defines a bevel angle of anchoring portion 33. Additionally, anchoring portion 33 define a tissue-separation angle (zeta) that is 30-50 degrees, e.g., 40 degrees.

Typically, tissue-cutting portion 42 extends 20-30% of a length L3 of body portion 34 of each tissue anchor 32. Length L3 of body portion 34 is typically between 1-5 mm. Each tissue-cutting portion 42 is configured to cut the tissue of the patient. Tissue-cutting portion 42 is shaped so as to define a pyramid shape of anchor 32 from pointed tip 36 toward converging points 48a and 48b. For some applications, tissue-cutting portion 42 is shaped so as to define a diamond shape of anchor 32 from pointed tip 36 toward converging points 48a and 48b. Anchoring portion 33 of each anchor 32 is shaped so as to define a tissue-expanding portion 44 disposed proximal to tissue-cutting portion 42. Each tissue-expanding portion 44 extends 70-80% of length L3 of body portion 34 of tissue anchor 32. Each tissue-expanding portion 44 is configured to expand but not cut the tissue of the patient.

Tissue-cutting portion 42 has a first height H1 of 0.3-0.7 mm (e.g., 0.5 mm) measured along central longitudinal axis 10 of tissue anchor 32. Tissue-expanding portion 44 has a second height H2 of 1.0-2.0 mm (e.g., 1.7 mm) measured along central longitudinal axis 10 of tissue anchor 32.

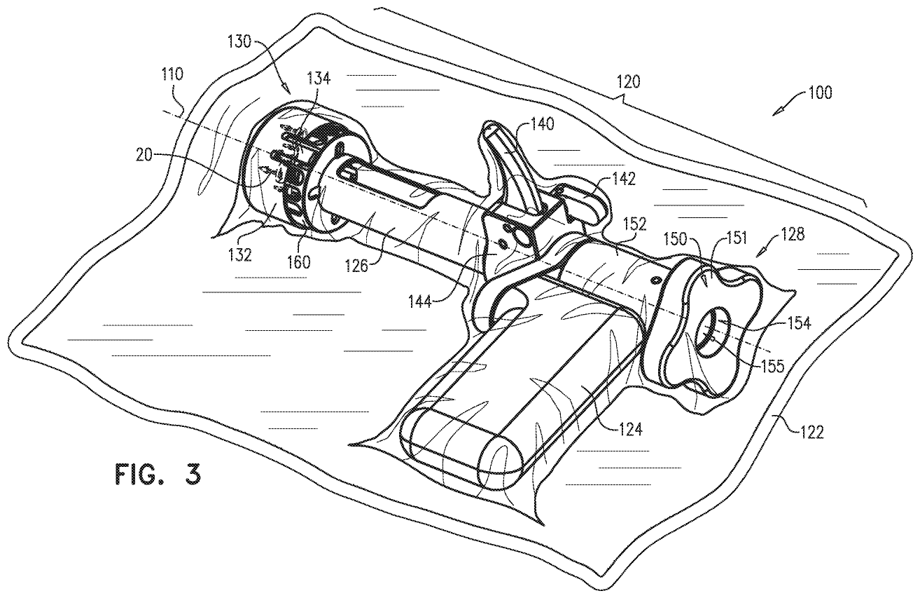

Reference is now made to FIG. 3, which is a schematic illustration of a system 100 comprising a delivery tool 120 for delivering and implanting closure device 20 described hereinabove with reference to FIGS. 1 and 2A-B, in accordance with some applications of the present invention. Delivery tool 120 comprises a handle 124 for a physician to hold during a medical procedure. Delivery tool 120 comprises an outer tube 126 that is disposed along a central longitudinal axis 110 of tool 120. Delivery tool 120 also a control element 150 that comprises a user interface (e.g., a knob, 151 as shown), at a proximal end portion 128 of tool 120. Knob 151 of control element 150 is petal shaped by way of illustration and not limitation. Knob 151 is moveable proximally along axis 110, as is described hereinbelow and moves proximally an outer cylindrical element 152. Additionally, rotation of knob 151 of control element 150 bidirectionally facilitates transitioning of closure device 20 between the resting and stretched states, as is described hereinbelow.

Typically, tool 120 comprises stainless steel 17-4PH per ASTM 899 and Polyphenylsulfone (PPSU).

Tool 120 is shaped so as to define an opening 154 at proximal end portion 128 of tool 120. Opening 154 facilitates passage of tools through a longitudinal lumen 155 of delivery tool 120, as is described hereinbelow.

Tool 120 comprises a trigger 140 which is coupled to a housing 144 that is rotatable around axis 110, as is described herein below. Trigger 140 is operatively associated with a closure-device holder at a distal end portion 130 of tool 120 as is described herein below. Pulling on trigger 140 facilitates firing of closure device 20 into tissue of the patient, as is described hereinbelow. A safety lock 142 is coupled to trigger 140 and, in a locked state of lock 142, trigger 140 cannot be pulled.

In FIG. 3, delivery tool 120 is shown in a packaging 122. In the packaged state, trigger 140 is disposed angularly with respect to handle 124 of tool 120. That is, in the packaged state, trigger 140 is not aligned with respect to handle 124. As is described hereinbelow, rotation of knob 151 of control element 150 (e.g., 5-90 rotational degrees) rotates housing 144 coupled to trigger 140 in order to align trigger 140 with handle 124 as is described hereinbelow.

Distal end portion 130 of tool 120 comprises a casing 160 within which closure device 20 is disposed. A protective cap 132 is coupled to casing 160 and comprises one or more radially-moveable legs 134 which help secure cap 132 to casing 160 and keep closure device 20 protected within casing 160. For some applications, cap 132 is transparent.

Reference is now made to FIGS. 4A-F and 5, which are schematic illustrations of operation of delivery tool 120 to transition closure device 20 described hereinabove with reference to FIGS. 1 and 2A-B between the resting and stretched states, in accordance with some applications of the present invention. FIG. 4A shows tool 120 in a state following removal from its packaging and prior to movement of any part of delivery tool. Distal end portion 130 of delivery tool 120 comprises casing 160 which houses a closure-device holder 180 that is configured to hold closure device 20. Holder 180 is moveable proximally and distally along longitudinal axis 110 of delivery tool 120. As is described hereinbelow, moving of holder 180 proximally moves proximally closure device 20 and loads holder 180 into a loaded state in order to facilitate subsequent firing distally of holder 180 in order to delivery (e.g., fire) closure device 20 into tissue of the patient.

As shown in view A, in the state following removal from its packaging and prior to movement of any part of delivery tool, holder 180 is disposed in its distal-most position. That is, holder 180 is disposed in a distal section of casing 160 such that anchoring portions 33 of tissue anchors 32 extend beyond a distal end 131 of tool (i.e., a distal end of casing 160) and into a space 135 provided by cap 132. Typically, casing 160 at distal end portion 130 defines distal end 131 of tool 120. In this distally-disposed state of holder 180, cap 132 protects the physician from accidental contact with anchoring portions 33 of closure device 20. Cap 132 is shaped so as to define radially-moveable legs 134 which are each shaped so as to define a respective protrusion 136 which is configured to fit within a respective window 162 of casing 160 and to protrude into a space provided by casing 160. When protrusions 136 are disposed within respective windows 162, cap 132 is securely coupled to delivery tool 120. As is described hereinabove, movement of legs radially away from axis 110 moves protrusions 136 out of windows 162 of casing 160 in order to facilitate decoupling of cap 132 from tool 120.

As shown in view A, holder 180 is coupled to a distal end of an inner tube 127 that slides within and with respect to outer tube 126. Inner tube 127 is coupled at a proximal end thereof to control element 150 at proximal end portion 128 of tool 120. As is described hereinbelow in FIG. 4C, knob 151 of control element 150 is pulled proximally so as to pull proximally inner tube 127 and thereby pull proximally holder 180. In the state following removal from its packaging and prior to movement of any part of delivery tool, trigger 140 is not aligned with handle 124 and is disposed angularly with respect to handle 124. In this state, knob 151 of control element 150 is restricted from being moved proximally so as to avoid any premature loading of holder 180. Only once housing 144 coupled to trigger 140 is rotated about axis 110 (e.g., typically by rotating knob 151 of control element 150 as is described hereinbelow), trigger 140 is aligned with handle 124, and knob 151 of control element 150 can be pulled proximally in order to pull holder 180 proximally into a loaded state. For some applications of the present invention, housing 144 is rotatable by the physician actively rotating housing 144.

Typically, holder 180 has a surface 181 against which closure device 20 is positioned. For some applications of the present invention, surface 181 is planar and has a plane that is disposed perpendicularly to longitudinal axis 110 of delivery tool 120. Holder 180 is shaped so as to define an opening 183 which is aligned with a central lumen 155 of tool 120. That is, lumen 155 extends from opening 154 at proximal end portion 128 of tool 120 and toward opening 183 at distal end portion 130 of delivery tool 120.

As shown in view B, delivery tool 120 comprises one or more (e.g., 4, as shown) closure-device-shape-adjusting elements 186 coupled to closure-device holder 180. Each closure-device-shape-adjusting element 186 is shaped so as to define a coupling 189 (e.g., a groove or an indentation) for removably coupling a portion of closure device 20 to closure-device-shape-adjusting element 186. Typically, each one of closure-device-shape-adjusting elements 186 is coupled to base 22 of closure device 20 at a respective outwardly-extending portion 26 of base 22 via coupling 189. Closure-device-shape-adjusting elements 186 are configured to adjust a shape of closure device 20 by being moveable radially inwardly and outwardly with respect to longitudinal axis 110. When elements 186 are moved outwardly away from longitudinal axis 110 of delivery tool 120, elements 186 facilitate transitioning of closure device 20 from the resting state toward the stretched state. When elements 186 are moved inwardly toward longitudinal axis 110 of delivery tool 120, elements 186 facilitate transitioning of closure device 20 from the stretched state toward the resting state.

For applications in which holder 180 comprises a planar surface 181, as shown, surface 181 is shaped so as to define one or more tracks 188 along which closure-device-shape-adjusting elements 186 move.

As shown in view B, in the state while delivery tool 120 is (1) within its packaging and (2) following removal from its packaging holder 180 and prior to movement of any part of delivery tool, closure device 20 is coupled to holder 180 and thereby to tool 120 in a resting state of closure device 20. Closure device 20 has a tendency to assume the resting state. That is, once closure device is stretched from its resting state and left untouched, it will automatically return to its resting state. Closure device 20 is coupled to tool 120 in its resting state so as to reduce strain on closure device 20.

In FIG. 4B, tool 120 is manipulated such that trigger 140 is rotated in a first rotational direction about central longitudinal axis 110 of tool 120 such that trigger 140 is aligned with handle 124. For some applications, trigger 140 is rotated 5-90 degrees, e.g., 45 degrees. For some applications, the physician rotates trigger 140 by touching trigger 140 and rotating it. For other applications, the physician touches housing 144 and rotates housing 144 in order to rotate trigger 140. For other applications, the physician touches knob 151 of control element 150 and rotates trigger 140 by rotating knob 151 of control element 150. Knob 151 of control element 150 is operatively coupled to housing 144 such that rotation of knob 151 rotates housing 144. Typically, control element 150 is coupled to outer cylindrical element 152, and rotating knob 151 of control element 150 rotates outer cylindrical element 152, responsively.

Knob 151 of control element 150 is coupled to an inner tube 127. Rotation of knob 151 in a first rotational direction rotates tube 127 in the first rotational direction. Rotation of tube 127 in the first rotational direction facilitates radial outward movement of closure-device-shape-adjusting elements 186 along their respective tracks 188. The radial outward movement of closure-device-shape-adjusting elements 186 pushes against outwardly-extending portions 26 so as to facilitate stretching of closure device 20 toward a stretched state. For some applications of the present invention, stretching of closure device 20 is controlled by the physician using control element 150. That is, the stretching of closure device 20 toward the stretched state may occur gradually, depending on the rate of actuation of control element 150 by the physician. In the stretched state of closure device 20, closure-device-shape-adjusting elements 186 are disposed in the radially-outmost position in the respective tracks 188, as shown.

That is, knob 151 is (a) gradually rotatable in a first rotational direction to move closure-device-shape-adjusting elements 186 radially outward in order to gradually transition closure device 20 from the resting state toward the stretched state, and (b) gradually rotatable in a second rotational direction to move closure-device-shape-adjusting elements 186 radially inward in order to gradually transition closure device 20 from the stretched state toward the resting state.

Typically, in order to transition closure device 20 from the resting state toward the stretched state, the physician rotates control element 150 5-90 degrees, e.g., 45 degrees.

Typically, in the stretched state, as shown in FIG. 4B, closure device 20 is more open and more stretched than in the resting state, as shown in FIG. 4A. That is, in the stretched state of closure device 20, and while closure device 20 is transitioned toward the stretched state from the resting state, a radially outward force is applied to the closure device by closure-device-shape-adjusting elements 186, and thereby, strain is applied to base 22 of closure device. In the stretched state of closure device 20 as shown in FIG. 4B, opening 21 has an stretched-state area of between 28 and 314 mm2, such as between 50 and 255 mm2, e.g., about 230 mm2. For some applications, the shape of opening 21 in the stretched state is similar to the shape of an asterisk or a flower having a plurality of petals. In the stretched state, opening 21 has a greatest distance Di4 between two opposite inwardly-extending portions 24 of 8-30 mm, such as 6-25 mm, e.g., 22 mm. Additionally, in the stretched state, closure device 20 has a greatest distance Di6 between any two opposite outwardly-extending portions 26 and including base 22 of 15-30 mm, e.g., 22 mm. For other applications, the shape is an ellipse, a square, another polygon (configuration not shown). For some applications a greatest distance Di3 across opening 21 of closure device 20 (i.e., not including the base) in the stretched state is 7-25 mm, e.g., 21 mm.

Reference is now made to FIGS. 1 and 4B, for some applications of the present invention, greatest distance Di3 across closure device 20 in the stretched state is 1-2 times greater, e.g., 1.3 times greater than greatest distance Di1 across closure device 20 in the resting state.

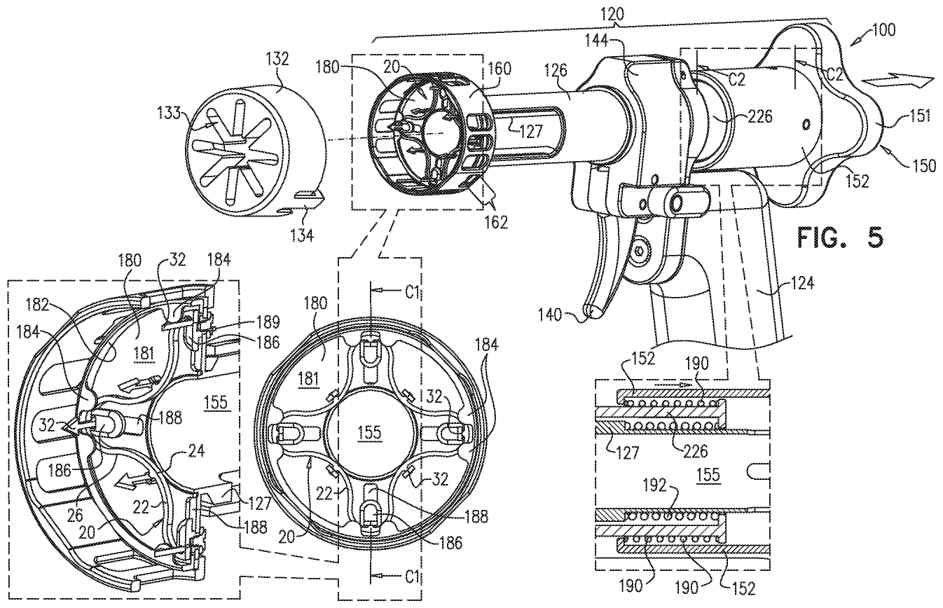

Reference is now made to FIGS. 4B and 5. As shown in view Cl of FIG. 5, closure-device holder 180 is shaped so as to define a rim 182 at a perimeter of a distal facing portion of holder 180, e.g., at a perimeter of surface 181. Rim 182 is shaped so as to define one or more securing tabs 184 which project radially toward a center of delivery tool 120. For example, for each one of anchors 32 at outwardly-extending portions 26 of closure device 20, holder 180 is shaped so as to provide two securing tabs 184 which are disposed at either side of each anchor 32 when closure device 20 is stretched to it stretched state. As shown by way of illustration and not limitation, holder 180 comprises 8 securing tabs 184. Each one of (e.g., each set of two) securing tabs 184 is configured to maintain coupling of closure device 20 to delivery tool 120 when a respective portion of closure device 20 (e.g., a portion of base 22 at outwardly-extending portion 26) is disposed in a space defined by (a) a portion of rim 182 of holder 180 of delivery tool 120, (b) a portion of surface 181 of closure-device holder 180, and (c) the one or more securing tabs 184.

Additionally, as described hereinabove, each of closure-device-shape-adjusting elements 186 is shaped so as to define a coupling 189 for housing a portion of base 22 of closure device 20 at outwardly-extending portions 26. In the stretched state of closure device 20, additional coupling of device 20 to holder 180 is achieved by friction between the respective portions of base 22 at outwardly-extending portions 26 and the respective closure-device-shape-adjusting elements 186 to which they are coupled.

Reference is now made to FIGS. 4B and 5. During the stretching of closure device 20 toward the stretched state, cap 132 remains coupled to tool 120. As shown in FIG. 5, cap 132 is shaped so as to define an opening 133. As shown, opening 133 of cap 132 is shaped so as to define a plurality of opening slots such that opening 133 is shaped so as to define an asterisk. The physician is able to visualize the stretching of closure device 20 within cap 132 via opening 133. The opening slots of opening 133 extend radially from a center of opening 133 such that the physician is able to see the radial motion of each of closure-device-shape-adjusting elements 186 and thereby of each respective anchor 32 coupled thereto, during the transitioning of closure device 20 from the resting state toward the stretched state. Additionally, for some applications of the present invention, cap 132 is transparent. It is to be noted that cap 132 is shown in FIG. 5 as not being coupled to casing 160 of tool 120 for clarity of illustration. It is to be noted that at this stage of use of tool 120, cap 132 is coupled to casing 160 of tool 120, as shown in FIG. 4B.

Tool 120 comprises a closure-device-holder-loading spring 192 which is operatively associated with closure-device holder 180 and is compressed so as to draw proximally holder 180 and closure device 20 coupled to holder 180 responsively to pulling proximally of control element 150, as is described hereinbelow. Spring 192 is disposed between inner tube 127 and cylindrical element 226 coupled to outer tube 126, at proximal end portion 128 of delivery tool 120. Control element 150 is moved proximally to compress a control-element-compressing spring 190 which is disposed between (a) cylindrical element 226 coupled to outer tube 126, and (b) outer cylindrical element 152. In order for the physician to move control element 150 proximally in order to move holder 180 into a loaded state, trigger 140 needs to be aligned with handle 124, as is shown in FIG. 4B. If trigger 140 is not aligned with handle 124 (e.g., as shown in FIGS. 3 and 4A), control element 150, and thereby holder 180, are restricted from being pulled proximally, and thereby, holder 180 is restricted from transitioning into a loaded state.

As shown in FIG. 4B, after the rotation of trigger 140 into alignment with handle 124, cap 132 remains coupled to casing 160 of tool 120.

Reference is now made to FIGS. 4C and 5. Control element 150 is pulled proximally, as indicated by the arrow at outer cylindrical element 152. As control element 150 is pulled proximally, cylindrical element 152 is pulled together with control element 150. The pulling proximally of control element 150 and cylindrical element 152 is accomplished by a distal end 153 of cylindrical element 152 pressing against control-element-compressing spring 190. Spring 190 is then compressed along longitudinal axis 110 of delivery tool 120 and compresses along an outer surface of cylindrical element 226 coupled to outer tube 126. Control-element-compressing spring 190 is compressed between a circular tab at distal end 153 of cylindrical element 152 and a circular tab at a proximal end 129 of cylindrical element 226 coupled to outer tube 126. As spring 190 is compressed between the two tabs, the compressed spring 190 between the two tabs pulls proximally outer tube 126 so as to longitudinally compress closure-device-holder-loading spring 192 in order to draw proximally closure-device holder 180 into a loaded state, as is described hereinbelow.

Once closure-device-holder-loading spring 192 is longitudinally compressed, spring 192 is locked in place, thereby locking closure-device holder 180 into the loaded state. Additionally, safety lock 142 maintains spring 192 in the compressed state and thereby, closure-device holder 180 in the loaded state. Lock 142 is later moveable into an unlocked state in order to release trigger 140, as is described hereinabove.

FIG. 4C is a representation of a brief moment in operation of tool 120, as control element 150 is subsequently released, thereby releasing cylindrical element 152, as is shown in FIG. 4D. In FIG. 4D, release of control element 150 by the physician releases the compressive force on a control-element-compressing spring 190 and spring 190 expands from its compressed state and toward a resting state of spring 190, as is shown in FIG. 4D. Responsively, control element 150 and cylindrical element 152 are moved distally and back into their resting places, as is shown in FIG. 4D. Additionally, distal end 153 of cylindrical element 152 returns to its original location against housing 144. It is to be noted that while control-element-compressing spring 190 is released to a non-compressed state, closure-device-holder-loading spring 192 remains in a compressed state and is locked in the compressed state by safety lock 142.

Knob 151 of control element 150 is coupled to a proximal end of inner tube 127. As shown in FIGS. 4C-D, compression of closure-device-holder-loading spring 192 draws proximally inner tube 127, and the proximal movement of inner tube 127 pulls proximally closure-device holder 180. Proximal movement of tube 127 moves proximally holder 180 within casing 160 and into a loaded state, as shown in FIG. 4D. Once holder 180 is disposed in the loaded state (i.e., in a proximal position within casing 160), pointed tips 36 of each anchor 32 are disposed within casing 160 and proximal to distal end 131 of tool 120. In this manner, closure device 20 is disposed entirely within casing 160, and casing 160 protects the physician from accidental contact with anchoring portions 33 of closure device 20, even after the removal of cap 132, as is described hereinbelow.

FIG. 4D shows the radial movement of radially-moveable legs 134 of cap 132 responsively to the drawing proximally of closure-device holder 180. Holder 180 has a circumference that is slightly smaller than a circumference of a wall of casing 160 such that holder 180 fits snugly within casing 160 while still being able to move proximally and distally. When holder 180 is moved proximally, the edges of holder 180 push against protrusions 136 of legs 134 forcing them to move radially outwardly with respect to central longitudinal axis 110 of tool 120. Movement of legs 134 moves protrusions 136 out of respective windows 162 of casing 160, thereby freeing legs 134 of cap from casing 160. Once protrusions 136 are not disposed within windows 162, cap 132 is in an unlocked state and can be removed by being pulled distally by the physician, as is shown in FIG. 4E.

It is to be noted that holder 180 moves proximally within casing 160, which remains stationary during the movement of holder 180. Casing has a distal end (i.e., distal end 131 of tool 120) which remains stationary during movement of closure-device holder 180 proximally and distally along longitudinal axis 110 of delivery tool 120.

FIG. 4E shows tool 120 with cap 132 removed therefrom. As shown, pointed tips 36 are disposed within casing 160 and proximal to distal end 131 of tool 120 in order to prevent accidental contact of tips 36 with skin of the physician.

It is to be noted that the stages of use of tool 120 as described hereinabove with reference to FIGS. 4A-E occur outside the body of the patient, before any procedure is performed on the patient. For some applications of the present invention, following the step shown in FIG. 4E (i.e., removal of cap 132), a medical procedure (e.g., a transcatheter aortic valve implantation (TAVI), transcutaneous mitral valve replacement (TMVR), or repair of any of the valves or other cardiac tissue) may be performed on a patient. For example, the physician creates an intercostal access to the pericardium, the physician uses a needle to puncture the myocardium and form a hole and passage therethrough. The physician advances a guidewire through the needle, withdraws the needle leaving the guidewire in the heart. For some applications, this is performed using the Seldinger technique. Then, once the steps performed in FIGS. 4A-E are performed, tool 120 is advanced over the guidewire.

Tool 120 is then centered with respect to the guide wire and is pushed distally until distal end 131 of tool 120, at the distal end of casing 160, touches the cardiac tissue. As is described hereinbelow, the physician unlocks trigger 140 and pulls on the trigger in order to fire holder 180 distally within casing 160 such that closure device 20 is exposed from within casing 160, and anchors 32 of closure device 20 are driven into the cardiac tissue in order to implant closure device within the tissue. Subsequently to the implanting of the closure device, a cardiac tool is advanced through opening 154 of tool 120 and through lumen 155 of tool 120 (and thereby also through opening 21 of closure device 20) in order to perform a procedure on the heart.

For some applications of the present invention, the cardiac tool is advanced through delivery tool 120 (and thereby also through opening 21 of closure device 20) and performs the procedure on the heart prior to implanting the implant in the cardiac tissue. Subsequently to the procedure, but before removal of the cardiac tool, the implant is implanted in the tissue, as is described hereinbelow.

In any case, following completion of the procedure performed on the heart, the cardiac tool is removed from the heart together with the guidewire and removed from within lumen 155 of tool 120. Subsequently, the hole made in the myocardium of the heart is closed by transitioning closure device 20, now implanted in the cardiac tissue, from the stretched state toward the resting state, as is described hereinbelow.

FIG. 4F shows tool 120 after trigger 140 has been pulled in order to fire holder 180 distally in order to implant closure device 20 in the cardiac tissue (not shown). As is described herein, safety lock 142 faces the distal direction which unlocks trigger 140 in order to enable the pulling of trigger 140. Responsively to the pulling of trigger 140 and the firing distally of holder 180, control-element-compressing spring 190 and closure-device-holder-loading spring 192 are both in a relaxed, non-compressed state.

Once closure device 20 is implanted in the tissue, closure device 20 is transitioned from its stretched state toward the resting state using tool 120. The physician rotates knob 151 of control element 150 in a second rotational direction that is opposite the first rotational direction described hereinabove with reference to FIG. 4B. Rotation of knob 151 of control element 150 rotates tube 127 in the second rotational direction. Rotation of tube 127 in the second rotational direction facilitates radial inward movement of closure-device-shape-adjusting elements 186 along their respective tracks 188. The radial inward movement of closure-device-shape-adjusting elements 186 moves outwardly-extending portions 26 radially inwardly so as to facilitate transitioning of closure device 20 toward the resting state. For some applications of the present invention, transitioning of closure device 20 toward the resting state is controlled by the physician using control element 150. That is, the transitioning of closure device 20 toward the resting state may occur gradually, depending on the rate of actuation of control element 150 by the physician. This gradual transition is shown in the transition of closure device from a stretched state shown in view A of FIG. 4F to a partially-stretched state, as shown in view B of FIG. 4F, and finally toward a resting state, as shown in view C of FIG. 4F. View B shows closure device 20 in a partially-stretched state that is 30-60%, e.g., 50%, between the stretched and resting states.

For some applications, as shown in view B of FIG. 4F, closure device 20 is configured to further assume a partially-stretched shape, in which opening 21 has a partially-stretched-shape area that is greater than the resting-state area and less than the stretched-state area, such as between 50% and 90% of the stretched-shape area, e.g., between 60% and 75% of the stretched-shape area. For example, the partially-stretched-shape area may be between 25 and 636 mm2, such as between 30 and 530 mm2. In the partially-stretched state, inwardly-extending portions 24 extend inwardly less than when closure device 20 assumes the resting state. The shape of closure device 20 in the partially-stretched state thus may be similar to the shape of an asterisk or a flower. For some applications, greatest distance Di7 across closure device 20 in the partially-stretched state is between 12 and 25 mm, e.g., 15 mm, and closest distance Dib between any two inwardly-extending portions 24 is between 3 and 16 mm, e.g., 9 mm.

As shown in view B, in the partially stretched state, the respective portions of closure device 20 (e.g., portions of base 22 at outwardly-extending portions 26) are moved from the spaces defined by (a) the respective portions of rim 182 of holder 180 of delivery tool 120, (b) the respective portions of surface 181 of closure-device holder 180, and (c) the respective securing tabs 184. When the portions of base 22 of closure device 20 are not disposed within these respective spaces, closure device 20 is initially decoupled from delivery tool 120. Closure device 20 is still held to tool 120 via friction between the respective portions of base 22 at outwardly-extending portions 26 and the respective closure-device-shape-adjusting elements 186 to which they are coupled.

As shown in view B, in the partially stretched state, each closure-device-shape-adjusting element 186 is disposed in a middle portion of its respective track 188.

Typically, in order to transition closure device 20 from the stretched state toward the resting state, the physician rotates control element 150 5-90 degrees, e.g., 45 degrees.

In response to rotation of control element 150 in the second rotational direction, trigger 140 is rotated about central longitudinal axis 110 of tool 120 in the second rotational direction such that trigger 140 is no longer aligned with handle 124. For some applications, trigger 140 is rotated 5-180 degrees. For some applications, the physician rotates trigger 140 by touching trigger 140 and rotating it. For other applications, the physician touches housing 144 and rotates housing 144 in order to rotate trigger 140. For other applications, the physician touches knob 151 of control element 150 and rotates trigger 140 by rotating knob 151 of control element 150. Knob 151 of control element 150 is operatively coupled to housing 144 such that rotation of knob 151 rotates housing 144. Typically, control element 150 is coupled to outer cylindrical element 152, and rotating knob 151 of control element 150 rotates outer cylindrical element 152, responsively.

Finally, in view C, closure device 20 assumes the resting state.

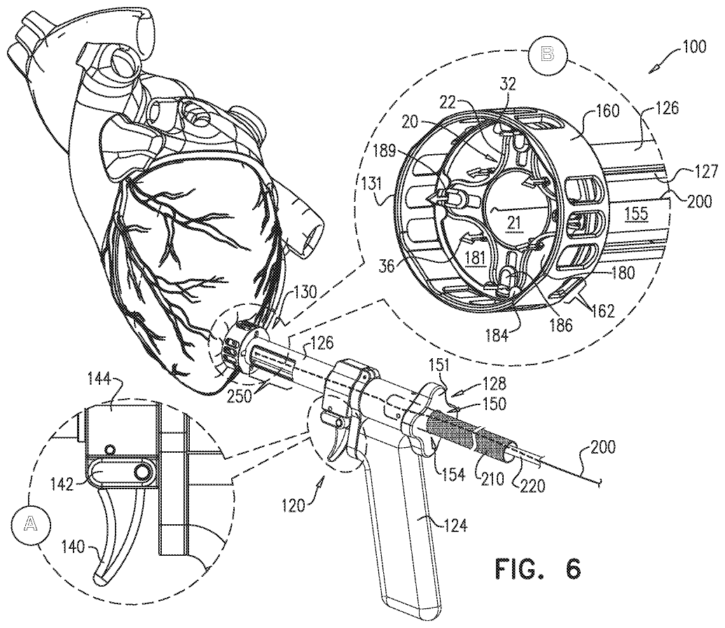

Reference is now made to FIGS. 6-9, which are schematic illustrations of delivery tool 20 described hereinabove with reference to FIGS. 3, 4A-F, and 5 being used to facilitate performing of a transapical surgical procedure and closure of a hole made in myocardial tissue, in accordance with some applications of the present invention. As described hereinabove, the stages of use of tool 120 as described hereinabove with reference to FIGS. 4A-E occur outside the body of the patient, before any procedure is performed on the patient. In these steps, safety lock 142 is in a locked state and faces in a proximal direction.

For some applications of the present invention, following the step shown in FIG. 4E (i.e., removal of cap 132), a medical procedure (e.g., a transcatheter aortic valve implantation (TAVI), transcutaneous mitral valve replacement (TMVR), or repair of any of the valves or other cardiac tissue) may be performed on the patient. For example, the physician creates an intercostal access to the pericardium, the physician uses a needle to puncture the myocardium and form a hole and passage therethrough. The physician advances a guidewire 200 through the needle, withdraws the needle leaving guidewire 200 in the heart. For some applications, this is performed using the Seldinger technique. Then, once the steps performed in FIGS. 4A-E are performed, tool 120 is advanced over guidewire 200.

In FIG. 6, distal end 131 of tool 120 is pushed against the cardiac tissue at the apical region of the heart, and tool 120 is centered with respect to guidewire 200. The physician is able to ensure that tool 120 is centered with respect to guidewire 200 by viewing guidewire 200 through a window 250 provided at distal portion 130 of tool 120. That is, tool 120 is shaped so as to provide window 250 at an opening defined by inner tube 127 and a corresponding opening defined by outer tube 260. At this stage, holder 180 is disposed in its loaded state and in a proximal position within casing 160 such that anchors 32 of closure device 20 are fully disposed within casing 160 and are disposed proximal to distal end 131 of tool 120.

A sheath 210 is introduced through opening 154 in proximal end portion 128 of tool 120 and partially advanced within lumen 155. Sheath 210 houses a cardiac tool 220 for performing a procedure on the heart, as described hereinabove. Sheath 210 and tool 220 are typically advanced around guidewire 200.

The physician then moves safety lock 142 into an unlocked state by moving safety lock 142 to face distally, and thereby trigger 140 is unlocked.

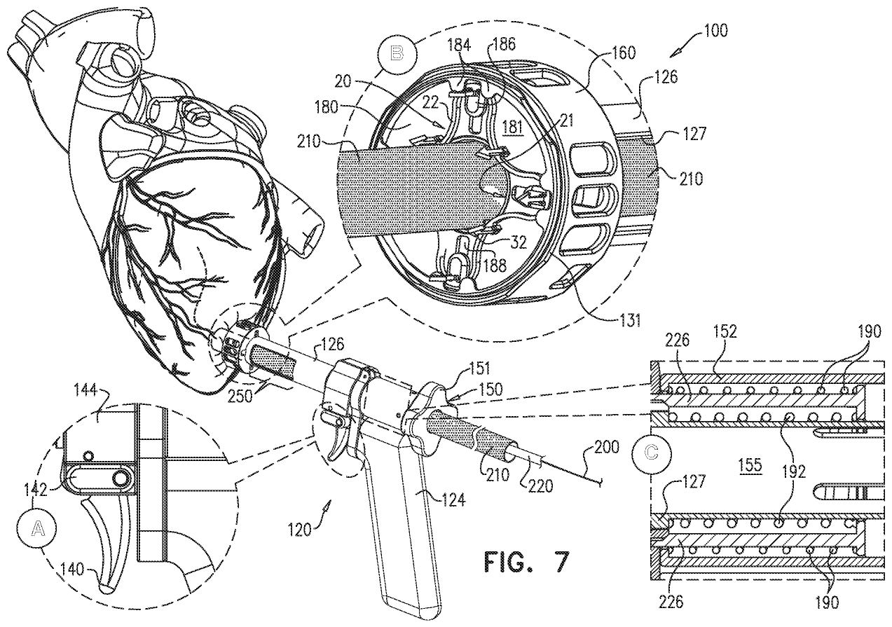

In FIG. 7, the physician pulls trigger 140, as shown in view A. Trigger 140 is operatively associated with spring 192. Trigger 140 is moveable to facilitate releasing of closure-device-holder-loading spring 192 from the compressed state and firing of closure-device holder 180 distally in order to fire closure device 20 distally and embed anchors 32 within the myocardial tissue, thereby implanting closure device 20 in the cardiac tissue. Pulling on trigger 140 releases closure-device-holder-loading spring 192, thereby causing expansion of spring 192 and distal movement of inner tube 127, as shown in view C. The distal movement of inner tube 127 moves distally closure-device holder 180, thereby firing closure device 20 into the tissue in order to implant closure device 20 within the tissue.

Once closure device 20 is implanted in the cardiac tissue, as shown in view B, the distal portions of sheath 210 and cardiac tool 220 are advanced distally further within lumen 155 of tool 120 and through opening 21 of closure device 20, and finally, into a heart chamber. (For clarity of illustrating how sheath 210 passes into the heart chamber, cardiac tissue is not shown in view B of FIG. 7. It is to be noted that at this stage, anchors 32 are embedded within the myocardial tissue.) The distal advancement of sheath 210 is viewed through window 250. The procedure (e.g., a transcatheter aortic valve implantation (TAVI), transcutaneous mitral valve replacement (TMVR), or repair of any of the valves or other cardiac tissue) is then performed on cardiac tissue. For applications in which replacement of a native valve (e.g., aortic, mitral, tricuspid, or pulmonary) of the patient is performed, cardiac tool 220 comprises a valve-replacement tool. For applications in which repair of a native valve (e.g., aortic, mitral, tricuspid, or pulmonary), repair of any other cardiac tissue (e.g., papillary muscles, chordae tendinae), or wall-to-wall adjustment is performed, cardiac tool 220 comprises a cardiac-repair tool.

The physician performs a medical procedure on the heart through delivery tool 120, and specifically through opening 21 of closure device 20. Typically, the medical procedure is performed on the heart while it is beating. For example, medical procedures that may be performed through tool 120 when inserted into the left ventricle include, but are not limited to:
valve replacement, such as aortic or mitral valve replacement;
valve repair, such as aortic or mitral valve repair;
left atrium ablation;
ascending, arch, and descending aortic stenting; and
left ventricle cardiac resynchronization therapy.

Medical procedures that may be performed through tool 120 when inserted into the right ventricle include, but are not limited to:
valve repair, such as tricuspid valve repair;
right heart ablation; and
pulmonary artery embolectomy.

For some applications of the present invention, the distal portions of sheath 210 and tool 220 are advanced prior to implanting closure device 20 in the myocardial tissue (configuration not shown). In this application of the present invention, closure device 20 is implanted immediately following the procedure on cardiac tissue performed by tool 220. That is, the distal portions of sheath 210 and tool 220 are advanced into the heart chamber immediately after the step performed in FIG. 6, but before closure device 20 is implanted in the myocardial tissue.

In either application of the present invention, after closure device 20 is implanted in the tissue and before closure device 20 is transitioned from the stretched state into the resting state, sheath 210, tool 220, and guidewire 200 are removed from within the heart and from within lumen 155 of tool 120. Only once sheath 210, tool 220, and guidewire 200 are removed, delivery tool 120 is used to transition closure device 20 from the stretched state into the resting state in order to facilitate closure of the hole made through the tissue of the myocardial wall.

Reference is now made to FIGS. 4F and 7. It is to be noted that the description of view A of FIG. 4F corresponds to the state of delivery tool 120 and closure device 20 in FIG. 7.

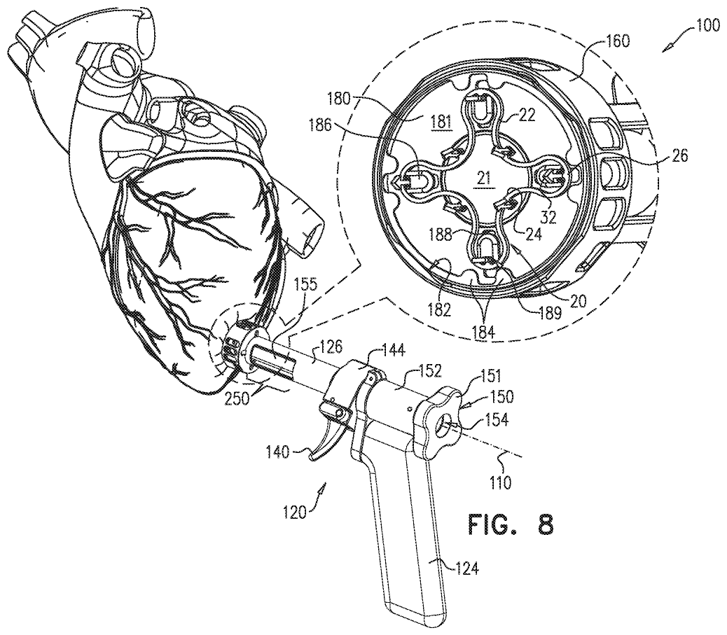

FIG. 8 shows partial closing of closure device 20 from the stretched state shown in FIG. 7 toward a resting state. As described hereinabove, knob 151 of control element 150 is rotated in the second rotational direction with respect to longitudinal axis 110 of tool 120 in order to transition closure device 20 toward the resting state. As knob 151 is rotated, trigger 140 and housing 144 rotate in the second rotational direction such that trigger is not aligned with handle 124 of tool 120, and tool 120 reverts toward its state in packaging 122, as shown in FIG. 3.

As closure device 20 transitions from the stretched state toward the resting state, anchors 32 move radially inwardly toward opening 21. Since anchors 32 of closure device 20 are embedded within the tissue of the myocardial wall of the heart, anchors 32 pull on the tissue during the transition of the closure device 20 from the stretched state toward the resting state. This pulls the tissue surrounding the hole inwardly toward a center of the hole, in order to close the hole.

Reference is now made to FIGS. 4F and 8. It is to be noted that the description of view B of FIG. 4F corresponds to the state of delivery tool 120 and closure device 20 in FIG. 8. As described hereinabove, even though the respective portions of closure device 20 (e.g., portions of base 22 at outwardly-extending portions 26) are moved from the spaces defined by (a) the respective portions of rim 182 of holder 180 of delivery tool 120, (b) the respective portions of surface 181 of closure-device holder 180, and (c) the respective securing tabs 184. When the portions of base 22 of closure device 20 are not disposed within these respective spaces, closure device 20 is initially decoupled from delivery tool 120. Closure device 20 is still held to tool 120 via friction between the respective portions of base 22 at outwardly-extending portions 26 and the respective closure-device-shape-adjusting elements 186 to which they are coupled.

As shown in FIG. 8, in the partially stretched state, each closure-device-shape-adjusting element 186 is disposed in a middle portion of its respective track 188.

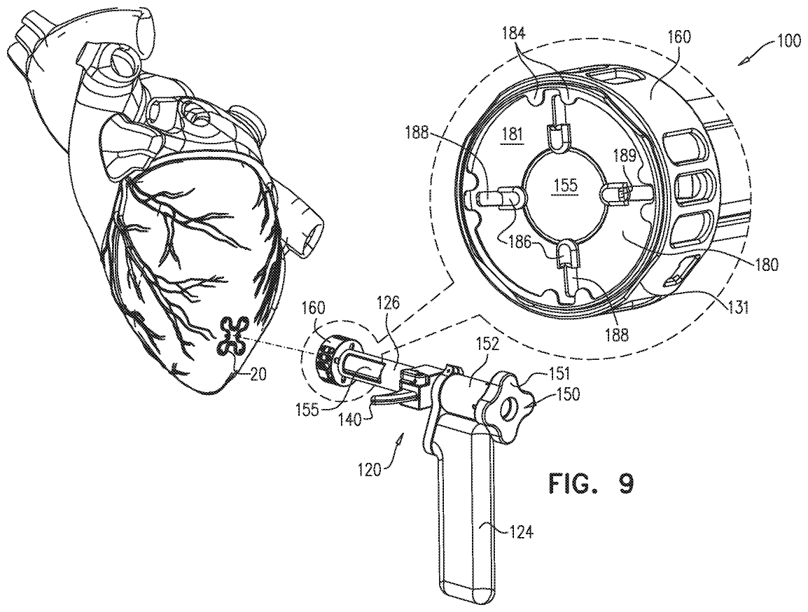

FIG. 9 shows closure device 20 in its resting state and implanted in the cardiac tissue. As shown, in the resting state, closure device 20 assumes a closed state in order to close the hole made in tissue of the myocardial wall of the heart. For some applications of the present invention, the physician additionally adds a stitch to prevent any blood leaking through the now-closed opening. Once delivery tool 120 has transitioned closure device 20 into the resting state, trigger 140 is not aligned with handle 124 and assumes its original position as it appeared in the packaged state, as described hereinabove with reference to FIG. 3. Additionally, lumen 155 of tool 120 is clear.

Once closure device 20 has been transitioned into the resting state, closure-device-shape-adjusting elements 186 are disposed in the radially-inmost position in the respective tracks 188, as shown. In order to fully decouple delivery tool 120 from closure device 20, tool 120 is pulled proximally to disengage holder 180 from closure device 20. Since, as described hereinabove, respective portions of base 22 of closure device 20 (e.g., portions of base 22 at outwardly-extending portions 26) are disposed within couplings 189 of closure-device-shape-adjusting elements 186. Generally in the partially-stretched and stretched states of closure device 20 friction is applied between the respective portions of base 22 and couplings 189 in order to maintain coupling of closure device 20 to holder 180. In the resting state of closure device 20, less friction is applied between the respective portions of base 22 and couplings 189. Once closure device 20 is implanted within the tissue, and anchors 32 prevent proximal motion of closure device 20, the closure device is decoupled from tool 120 when the physician pulls proximally on tool 120. That is, the respective portions of base 22 are decoupled from couplings 189.

Reference is now made to FIGS. 1, 4A, and 9. It is to be noted that the description closure device 20 of FIGS. 1 and 4A corresponds to the state of closure device 20 in FIG. 9.

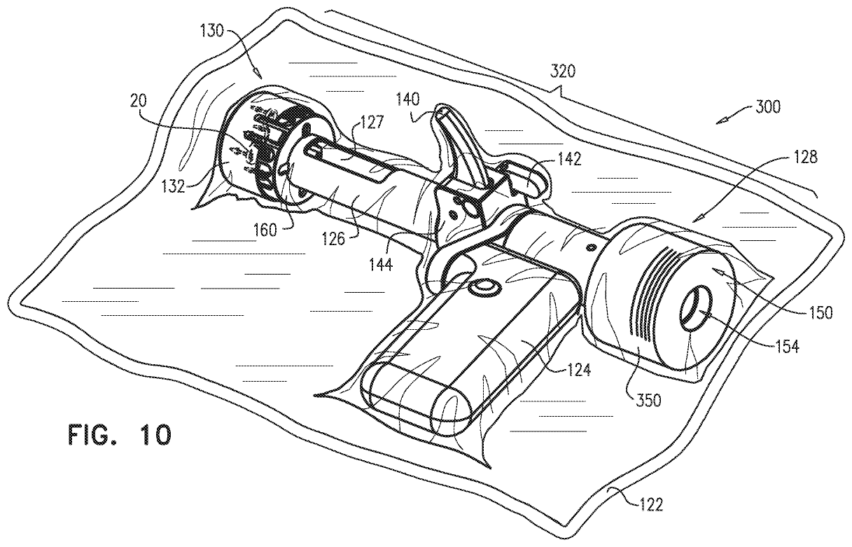

Reference is now made to FIG. 10, which is a schematic illustration which is a schematic illustration of a system 300 comprising a delivery tool 320 for delivering and implanting closure device 20 described hereinabove, in accordance with some applications of the present invention. It is to be noted that delivery tool 320 is similar to delivery tool 120 described herein with reference to FIGS. 3-9, with the exception of control element 150 comprising a motor 350 which automatically performs the rotation of control element 150 in first and second rotational direction in order to bidirectionally rotate trigger 140 and also transition closure device 20 between the resting and stretched states, as described hereinabove. Typically, motor 350 gradually and smoothly controls the transition of closure device 20 between the resting and stretched states.

That is, motor 350 is (a) automatically actuatable to move more closure-device-shape-adjusting elements 186 radially outward in order to gradually transition closure device 20 from the resting state toward the stretched state, and (b) automatically actuatable to move closure-device-shape-adjusting elements 186 radially inward in order to gradually transition closure device 20 from the stretched state toward the resting state.

Reference is now made to FIGS. 1-10. It is to be noted that closure device 20 shown in FIGS. 3-10 is identical to closure device 20 shown in FIG. 1, i.e., ground as described in FIGS. 2A-B. That is anchoring portions 33 of closure device 20 of FIGS. 3-10 are the same as anchoring portions 33 of closure device 20 shown in FIG. 1, i.e., ground as described in FIGS. 2A-B.

Reference is now made to FIGS. 1-10. It is to be noted that closure device 20 and delivery tools 120 and 320 may be used on tissue other than cardiac tissue of the patient. That is, closure device 20 may be used to close a hole made in any tissue of the body. For example, closure device 20 and tools 120 and 320 may be used to close a hole made in skin of the patient. For other applications, closure device 20 and tools 120 and 320 may be used to close a hole made in gastric tissue of the patient. Additionally, it is to be noted that systems 100 and 300 used to describe positioning of closure device in a transapical procedure may be used on procedures performed on the heart other than a transapical procedure. For example, delivery tools 120 and 320 can be positioned anywhere along the heart and not just at the apex. Similarly, closure device 20 can be implanted in tissue other than myocardial tissue at or near the apex.

For some applications of the present invention, techniques and apparatus described in U.S. Pat. No. 9,282,954 to Bolotin are combined with techniques and apparatus described herein.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus, comprising:
an implantable cardiac closure device having a stretched state and a resting state, the closure device being configured for facilitating closure of a hole made through tissue of a myocardial wall of a patient; and
a delivery tool comprising:
a closure-device holder, at a distal portion of the delivery tool, being configured to hold the closure device, the closure-device holder being moveable proximally and distally along a longitudinal axis of the delivery tool; and
one or more closure-device-shape-adjusting elements coupled to the closure-device holder, the one or more closure-device-shape-adjusting elements each being shaped so as to define a coupling for removably coupling the closure device to the one or more closure-device-shape-adjusting elements, the one or more closure-device-shape-adjusting elements being configured to adjust a shape of the closure device by being moveable radially inwardly toward the longitudinal axis of the delivery tool to transition the closure device from the stretched state toward the resting state.

2. The apparatus according to claim 1, wherein the one or more closure-device-shape-adjusting elements are configured to further adjust the shape of the closure device by being moveable radially outwardly away from the longitudinal axis of the delivery tool to transition the closure device from the resting state toward the stretched state.

3. The apparatus according to claim 1, wherein the distal portion of the delivery tool comprises a casing within which the closure-device holder moves proximally and distally, the casing having a distal end which remains stationary during movement of the closure-device holder proximally and distally along the longitudinal axis of the delivery tool.

4. The apparatus according to claim 3, wherein, when the holder is moved proximally, the closure device is disposed entirely within the casing.

5. The apparatus according to claim 3, further comprising a cap reversibly coupled to the casing, wherein the cap protects the closure device when the closure device and the closure-device holder are disposed at a distal portion of the casing.

6. The apparatus according to claim 5, wherein, when the closure device is disposed at a distal portion of the casing, tissue anchors of the closure device extend beyond the distal end of the casing.

7-8. (canceled)

9. The apparatus according to claim 1, further comprising a control element at a proximal portion of the delivery tool, the control element being operatively associated with the one or more closure-device-shape-adjusting elements in order to gradually move the one or more closure-device-shape-adjusting elements radially in order to gradually transition the closure device between the stretched and resting states.

10-16. (canceled)

17. The apparatus according to claim 1, wherein the delivery tool further comprises:
a spring operatively associated with the closure-device holder and compressible proximally along the longitudinal axis of the delivery tool into a compressed state to draw proximally the closure-device holder into a loaded state; and
a trigger operatively associated with the spring and moveable to facilitate releasing of the spring from the compressed state and firing of the closure-device holder distally.

18. (canceled)

19. The apparatus according to claim 1, wherein:
the closure device is coupled to the delivery tool in the resting state,
the delivery tool is configured to:
(a) transition the closure device into the stretched state,
(b) subsequently, deliver the closure device into the tissue of the myocardial wall surrounding the hole made through the tissue, while the closure device is in the stretched state, and
(c) subsequently, transition the closure device from the stretched state toward the resting state to facilitate closure of the hole in the tissue.

20. The apparatus according to claim 19, wherein the closure device comprises a superelastic material.

21-25. (canceled)

26. A tissue anchor for implantation in tissue of a patient, the tissue anchor comprising:
a pointed tip at a distal end of the tissue anchor;
a barbed surface proximal to the pointed tip and configured to prevent proximal movement of the tissue anchor following penetration of the tissue anchor in the tissue of the patient;
a body portion extending between the pointed tip and the barbed surface;
a tissue-cutting portion extending directly proximal from the pointed tip, the tissue-cutting portion extending 20-30% of a length of the body portion of the tissue anchor and configured to cut the tissue of the patient; and
a tissue-expanding portion disposed proximal to the tissue-cutting portion, the tissue-expanding portion extending 70-80% of the length of the body portion of the tissue anchor and configured to expand but not cut the tissue of the patient.

27-33. (canceled)

34. The tissue anchor according to claim 26, wherein the tissue-cutting portion comprises at least one cutting edge.

35. (canceled)

36. The tissue anchor according to claim 34, wherein the tissue-cutting portion comprises two cutting edges that are disposed symmetrically with respect to a central longitudinal axis of the tissue anchor.

37. The tissue anchor according to claim 34, wherein:
the at least one cutting edge comprises a first cutting edge,
the body portion comprises a first flat surface that extends from a first edge of the barbed surface and distally toward a first converging point at the first flat surface,
the first converging point is disposed distal to the first edge of the barbed surface,
the first converging point defines an end of the first cutting edge,
the first cutting edge extends angularly between the first converging point and the pointed distal tip, and
the first flat surface has a first-flat-surface plane that is disposed in parallel with a central longitudinal axis of the tissue anchor at the pointed distal tip.

38-46. (canceled)

47. A method, comprising:
providing an implantable cardiac closure device and a delivery tool, the delivery tool including:
a closure-device holder at a distal portion of the delivery tool, the closure-device holder being configured to hold the closure device, the closure-device holder being moveable proximally and distally along a longitudinal axis of the delivery tool; and
one or more closure-device-shape-adjusting elements coupled to the closure-device holder, the one or more closure-device-shape-adjusting elements each being shaped so as to define a coupling for removably coupling the closure device to the one or more closure-device-shape-adjusting elements; and
using the delivery tool:
implanting the closure device in a body of a patient in tissue of a myocardial wall surrounding a hole made through the tissue; and
facilitating closing of the hole by adjusting a shape of the closure device, by moving the one or more closure-device-shape-adjusting elements radially inwardly toward the longitudinal axis of the delivery tool, thereby transitioning the closure device from a stretched state toward a resting state.

48. The method according to claim 47, further comprising, subsequently to the implanting of the closure device, and prior to the facilitating of the closing of the opening in the tissue, performing replacement of a native cardiac valve of the patient by advancing a valve-replacement tool through the closure device, the valve-replacement tool being configured for replacing the native cardiac valve.

49. The method according to claim 47, further comprising, subsequently to the implanting of the closure device, and prior to the facilitating of the closing of the opening in the tissue, performing repair of cardiac tissue of the patient by advancing a cardiac-repair tool through the closure device, the cardiac-repair tool being configured for repairing the cardiac tissue.

50. (canceled)

51. The method according to claim 47, further comprising, prior to the implanting of the closure device, moving the closure-device holder proximally along the longitudinal axis of the delivery tool to draw proximally the closure-device holder and the closure device.

52-56. (canceled)

57. The method according to claim 47, wherein the closure-device holder has a surface against which the closure device is positioned.

58-60. (canceled)

61. The method according to claim 47, wherein:
the delivery tool includes a control element including a user interface, the control element being operatively associated with the one or more closure-device-shape-adjusting elements,
moving the one or more closure-device-shape-adjusting elements radially inwardly comprises gradually moving the one or more closure-device-shape-adjusting elements by actuating the control element, and
by the gradually moving the one or more closure-device-shape-adjusting elements radially inwardly, gradually transitioning the closure device toward the resting state.

62-88. (canceled)
